(19) United States
(12) Patent Application Publication
(10) Pub. No.: US 2024/0293228 A1
(43) Pub. Date: Sep. 5, 2024

(54) PROSTHETIC VALVES WITH WIDER OUTFLOW CELLS

(71) Applicant: EDWARDS LIFESCIENCES CORPORATION, Irvine, CA (US)

(72) Inventor: Tamir S. Levi, Zikhron Yaakov (IL)

(21) Appl. No.: 18/662,950

(22) Filed: May 13, 2024

Related U.S. Application Data
(63) Continuation of application No. PCT/US2022/049864, filed on Nov. 14, 2022.
(60) Provisional application No. 63/279,299, filed on Nov. 15, 2021.

(51) Int. Cl. A61F 2/24; A61F 2/00

(52) U.S. Cl. CPC A61F 2/2418; A61F 2002/0081; A61F 2250/001

(57) ABSTRACT

The present disclosure relates to prosthetic valves with expandable frames. As one example, a prosthetic valve can include a frame comprising a first row comprising a plurality of wide cells, a second row comprising a plurality of narrow cells, and a third row a plurality of narrow cells. Each wide cell of the first row can be directly coupled to two other adjacent wide cells of the first row, and each narrow cells of any of the second and third rows can be directly coupled to adjacent narrow cells of the same rows. The width of each wide cells of the first row can be at least twice as great as the width of any narrow cell.

### CROSS REFERENCE TO RELATED APPLICATIONS

This application is a continuation of International Application No. PCT/US2022/049864, filed Nov. 14, 2022, which claims benefit of U.S. Provisional Application No. 63/279,299, filed on Nov. 15, 2021, the contents of each of which are herein incorporated by reference in their entirety.

### FIELD

The present disclosure relates to prosthetic heart valves, and in particular, to prosthetic valves with frames having a first row of outflow cells which are wider than cells of the second and third rows.

### BACKGROUND

Native heart valves, such as the aortic, pulmonary and mitral valves, function to assure adequate directional flow from and to the heart, and between the heart's chambers, to supply blood to the whole cardiovascular system. Various valvular diseases can render the valves ineffective and require replacement with artificial valves. Surgical procedures can be performed to repair or replace a heart valve. Surgeries are prone to an abundance of clinical complications, hence alternative less invasive techniques of delivering a prosthetic heart valve over a catheter and implanting it over the native malfunctioning valve, have been developed over the years.

Different types of prosthetic heart valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, towards the native malfunctioning valve. Once the prosthetic valve is properly positioned at the desired site of implantation, it can be expanded against the surrounding anatomy, such as an annulus of a native valve, and the delivery assembly can be retrieved thereafter.

### SUMMARY

A prosthetic valve includes a leaflet assembly coupled to a support frame that includes intersecting struts, defining rows of closed cells. Some of these cells may be facing the coronary ostia in the case of aortic valve replacement. In some instances, a patient may require implantation of a coronary stent or other procedure that requires access to a coronary artery after prosthetic valve implantation. For such instances, a physician may need to access the coronary artery through an opening defined by a cell facing the coronary ostium. Accordingly, a need exists for improved prosthetic valves designed to provide adequate access to the coronary arteries therethrough.

The present disclosure is directed towards prosthetic valve comprising an expandable frame having several rows of cells, such as a first cell row, a second cell row, and a third cell row, wherein the first cell row includes a plurality of wide cells which are wider than narrow cells of the second and third rows.

According to some aspects of the disclosure, there is provided a prosthetic valve comprising a frame having an inflow end and an outflow end, wherein the frame is movable between a radially compressed and a radially expanded state. The frame comprises a first cell row comprising a plurality of wide cells, wherein each wide cell defines an outflow apex and comprises two second-length proximal struts diverging therefrom. The frame further comprises a second cell row comprising a plurality of narrow cells. The frame further comprises a third cell row comprising a plurality of narrow cells.

In some examples, each wide cell of the first cell row is directly coupled to two other adjacent wide cells of the first cell row.

In some examples, each narrow cell of the second cell row is directly coupled to two adjacent narrow cells of the second cell row.

In some examples, each narrow cell of the third cell row is directly coupled to two adjacent narrow cells of the third cell row.

In some examples, the number of narrow cells in the second cell row is identical to the number of narrow cells in the third cells row, wherein all of the narrow cells of the second cell row and the third cell row have the same height and the same width.

In some examples, the width of each wide cell of the first cell row is at least twice as great as the width of any narrow cell.

In some examples, the width of each wide cell of the first cell row is at least three times as great as the width of any narrow cell.

According to some aspects of the disclosure, there is provided a prosthetic valve comprising a frame having an inflow end and an outflow end, wherein the frame is movable between a radially compressed and a radially expanded state. The frame comprises a first cell row comprising a plurality of cells, which comprise a plurality of wide cells and a plurality of narrow cells, wherein each wide cell defines a first outflow apex and comprises two second-length proximal struts diverging therefrom, wherein each narrow cell of the first cell row defines a second outflow apex and comprises two first-length proximal struts diverging therefrom. The frame further comprises a second cell row comprising a plurality of narrow cells. The frame further comprises a third cell row comprising a plurality of narrow cells. The width of each wide cell of the first cell row is at least twice as great as the width of any narrow cell.

In some examples, each narrow cell of the second cell row is directly coupled to two adjacent narrow cells of the second cell row.

In some examples, each narrow cell of the third cell row is directly coupled to two adjacent narrow cells of the third cell row.

In some examples, each cell of the first cell row is directly coupled to two other adjacent cells of the first cell row.

In some examples, the number of narrow cells in the second cell row is identical to the number of narrow cells in the third cells row, wherein all of the narrow cells of the first cell row, the second cell row and the third cell row, have the same height and the same width.

According to some aspects of the disclosure, there is provided a prosthetic valve comprising a frame having an inflow end and an outflow end, wherein the frame is movable between a radially compressed and a radially expanded state. The frame comprises a first cell row comprising three wide cells, wherein each wide cell defines an outflow apex and comprises two second-length proximal struts diverging therefrom, and wherein each wide cell of the first cell row is directly coupled to two other adjacent wide cells of the first cell row. The frame further comprises a second cell row comprising six narrow cells, wherein each narrow cell of the second cell row is directly coupled to two adjacent narrow cells of the second cell row. The frame further comprises a third cell row comprising six narrow cells, wherein each narrow cell of the third cell row is directly coupled to two adjacent narrow cells of the third cell row. All of the narrow cells of the second cell row and the third cell row have the same height and the same width, wherein the width of each wide cell of the first cell row is at least twice as great as the width of any narrow cell.

According to some aspects of the disclosure, there is provided a prosthetic valve comprising a frame having an inflow end and an outflow end, wherein the frame is movable between a radially compressed and a radially expanded state. The frame comprises a first cell row comprising nine wide cells, wherein each wide cell defines an outflow apex and comprises two second-length proximal struts diverging therefrom, and wherein each wide cell of the first cell row is directly coupled to two other adjacent wide cells of the first cell row. The frame further comprises a second cell row comprising eighteen narrow cells, wherein each narrow cell of the second cell row is directly coupled to two adjacent narrow cells of the second cell row. The frame further comprises a third cell row comprising eighteen narrow cells, wherein each narrow cell of the third cell row is directly coupled to two adjacent narrow cells of the third cell row. All of the narrow cells of the second cell row and the third cell row have the same height and the same width, wherein the width of each wide cell of the first cell row is at least twice as great as the width of any narrow cell.

According to some aspects of the disclosure, there is provided a prosthetic valve comprising a frame having an inflow end and an outflow end, wherein the frame is movable between a radially compressed and a radially expanded state. The frame comprises a first cell row comprising a plurality of wide cells, wherein each wide cell defines an outflow apex and comprises two second-length proximal struts diverging therefrom, and wherein each wide cell of the first cell row is directly coupled to two other adjacent wide cells of the first cell row. The frame further comprises a second cell row comprising a plurality of narrow cells, wherein each narrow cell of the second cell row is directly coupled to two adjacent narrow cells of the second cell row, and wherein each narrow cell of the second cell row comprises two first-length proximal struts converging at a free-ended proximal junction. The frame further comprises a third cell row comprising a plurality of narrow cells, wherein each narrow cell of the third cell row is directly coupled to two adjacent narrow cells of the third cell row.

In some examples, the number of narrow cells in the second cell row is identical to the number of narrow cells in the third cells row, wherein all of the narrow cells of the second cell row and the third cell row have the same height and the same width.

In some examples, the width of each wide cell of the first cell row is at least twice as great as the width of any narrow cell.

In some examples, the width of each wide cell of the first cell row is at least three times as great as the width of any narrow cell.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE FIGURES

Some examples of the disclosed technology are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the disclosed technology. For the sake of clarity, some objects depicted in the figures are not to scale.

In the Figures:

FIG. 1A shows a perspective view of a prosthetic valve, according to some examples.

FIG. 1B shows a perspective view of a frame of the prosthetic valve of FIG. 1A.

FIG. 2 shows a side elevation view of the frame of FIG. 1B, depicting the valve frame in a flat configuration.

FIG. 3 shows a side elevation view a frame with three wide cells in the first cell row and six narrow cells in each of the second and third cell rows, according to some examples.

FIG. 4 shows a side elevation view a frame with three wide cells in the first cell row and nine narrow cells in each of the second and third cell rows, according to some examples.

FIG. 5 shows a side elevation view a frame with three wide cells in the first cell row and twelve narrow cells in each of the second and third cell rows, according to some examples.

FIGS. 6A-D shows side elevation views of exemplary frames with three wide cells in the first cell row and fifteen narrow cells in each of the second and third cell rows.

FIG. 7 shows a side elevation view a frame with three wide cells in the first cell row and eighteen narrow cells in each of the second and third cell rows, according to some examples.

FIG. 8 shows a side elevation view a frame with six wide cells in the first cell row and eighteen narrow cells in each of the second and third cell rows, according to some examples.

FIG. 9 shows a side elevation view a frame with nine wide cells in the first cell row and eighteen narrow cells in each of the second and third cell rows, according to some examples.

FIG. 10 shows a side elevation view a frame with three wide cells and six narrow cells in the first cell row, according to some examples.

FIG. 11 shows a side elevation view a frame with three wide cells and nine narrow cells in the first cell row, according to some examples.

FIG. 12 shows a side elevation view a frame with six wide cells and three narrow cells in the first cell row, according to some examples.

FIG. 13 shows a side elevation view a frame with three wide cells in the first cell row, six narrow cells in each of the second and third cell rows, and three narrow cells in an intermediate cell row, according to some examples.

FIG. 14 shows a side elevation view a frame with three wide cells and six narrow cells in the first cell row, twelve narrow cells in each of the second and third cell rows, and nine narrow cells in an intermediate cell row, according to some examples.

FIG. 15 shows a side elevation view a frame with three wide cells in the first cell row and six narrow cells in each of the second and third cell rows, according to some examples.

FIG. 16 shows a side elevation view a frame with six wide cells in the first cell row and twelve narrow cells in each of the second and third cell rows, according to some examples.

FIG. 17 shows a perspective view of a delivery assembly comprising a delivery apparatus carrying a prosthetic valve, according to some examples.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed towards all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises.” As used herein, “and/or” means “and” or “or,” as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

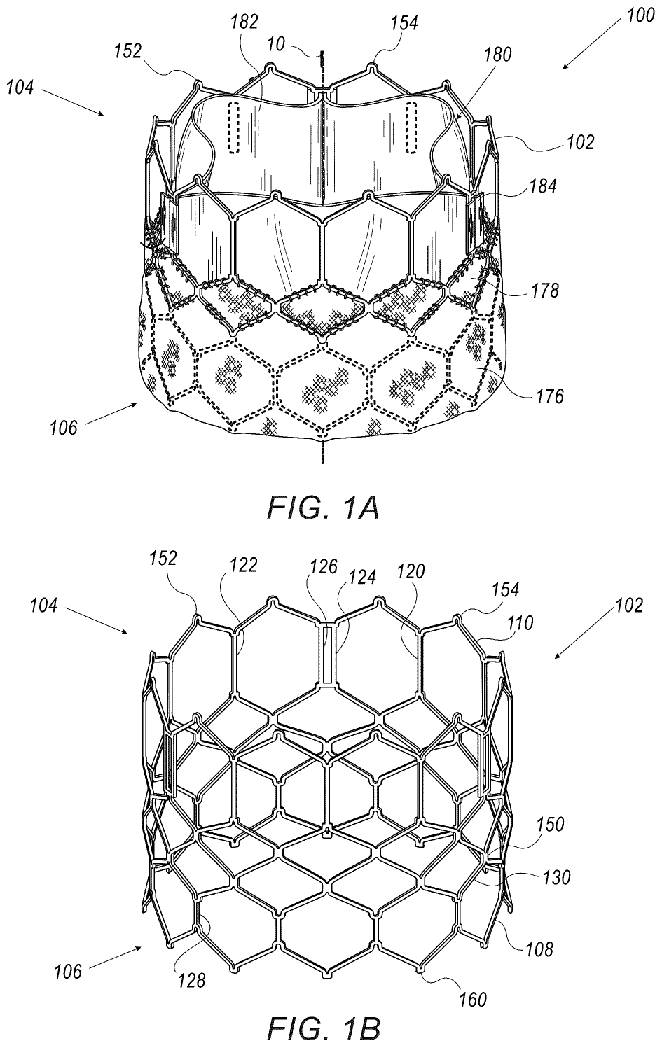

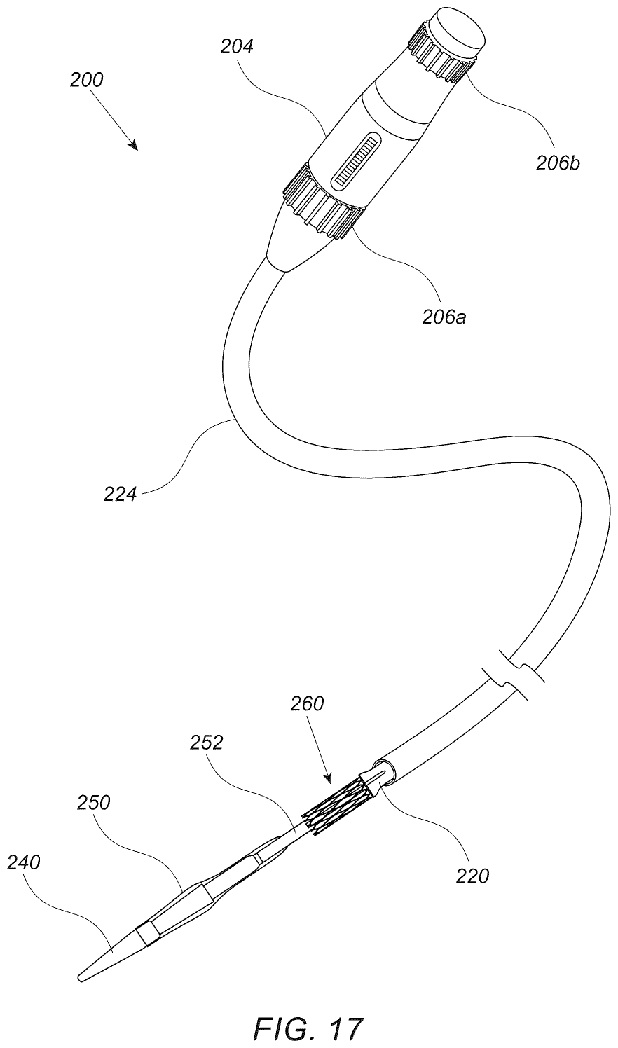

FIGS. 1A and 1B show perspective views of an example of a prosthetic valve 100, with and without soft components (such as skirts and a leaflet assembly), respectively. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valves can be crimped on or retained by an implant delivery apparatus 200 (see FIG. 17) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve 100 of the current disclosure may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus 200 (see FIG. 17). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165 and U.S. Provisional Application No. 63/085,947, filed Sep. 30, 2020, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

The term “plurality”, as used herein, means more than one.

The prosthetic valve 100 comprises an inflow end 106 and an outflow end 104. In some instances, the inflow end 106 is the distal end of the prosthetic valve 100, and the outflow end 104 is the proximal end of the prosthetic valve 100. Alternatively, depending for example on the delivery approach of the valve, the inflow end can be the proximal end of the prosthetic valve, and the outflow end can be the distal end of the prosthetic valve.

The term “proximal”, as used herein, generally refers to a position, direction, or portion of a device or a component of a device, which is closer to the user (for example, during an implantation procedure) and further away from the implantation site.

The term “distal”, as used herein, generally refers to a position, direction, or portion of a device or a component of a device, which is further away from the user and closer to the implantation site.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

The terms “longitudinal” and “axial”, as used herein, refer to an axis extending in the proximal and distal directions, unless otherwise expressly defined.

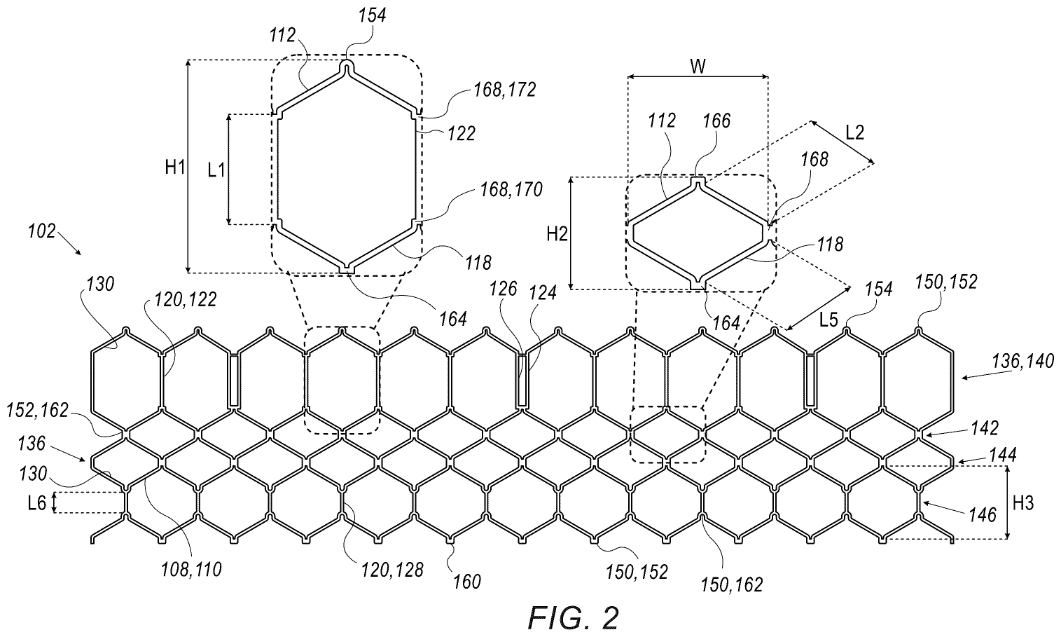

The valve 100 comprises an annular frame 102 movable between a radially compressed state and a radially expanded state, and a leaflet assembly 180 mounted within the frame 102. FIG. 2 shows the frame 102 in a flat configuration for purposes of illustration. The frame 102 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter (not shown), and then expanded inside a patient by an inflatable balloon or equivalent expansion mechanism. Alternatively or additionally, the frame 102 can be made of shape-memory materials such as, but not limited to, nickel titanium alloy (e.g., Nitinol).

In the example illustrated in FIGS. 1A-1B, the frame 102 is an annular, stent-like structure comprising a plurality of intersecting struts 108, and defining a central axis 10. In this application, the term “strut” 108 encompasses vertical struts, angled or curved struts, support posts, commissure windows, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define a plurality of cells 130 arranged in several cell rows 136. The frame 102 can have a cylindrical or substantially cylindrical shape having a constant diameter from the inflow end 106 to the outflow end 104 as shown, or the frame can vary in diameter along the height of the frame, as disclosed in U.S. Pat. No. 9,155,619, which is incorporated herein by reference.

Struts 108 comprise angled struts 110, and optionally vertical struts 120. The term “vertical strut” refers to a strut that generally extends in an axial direction, while the term “angled strut” generally refers to a strut that can extend at an angle relative to an axial line intersecting therewith along a plane defined by the frame 102. It is to be understood that the term “angled strut” encompasses both linear angled struts and curved struts.

FIGS. 1A-1B show an exemplary prosthetic valve 100 that can be representative of, but is not limited to, a balloon expandable prosthetic valve. The frame 102 of the prosthetic valve 100 illustrated in FIGS. 1B and 2 comprises four cell rows 136, namely a first cell row 140 which can be also referred to as the upper cell row, a second cell row 142, a third cell row 144, and a fourth cell row 146. The four rows of cells are shown by way of illustration and not limitation, and it is to be understood that more or cells rows 136 may be defined by other frame configurations. Zoomed-in view of a cell of the first cell row 140 and a cell of the second cell row 142 are further illustrated in FIG. 2.

Two or more struts 108 can intersect at junctions 150, which can be can be equally or unequally spaced apart from each other. The struts 108 may be pivotable or bendable relative to each other, so as to permit frame expansion or compression. For example, the frame 102 can be formed from a single piece of material, such as a metal tube, via various processes such as, but not limited to, laser cutting, electroforming, and/or physical vapor deposition, while retaining the ability to collapse/expand radially in the absence of hinges and like.

The leaflet assembly 180 comprises a plurality of leaflets 182 (e.g., three leaflets), positioned at least partially within the frame 102, and configured to regulate flow of blood through the prosthetic valve 100 from the inflow end 106 to the outflow end 104. While three leaflets 182 arranged to collapse in a tricuspid arrangement, are shown in the example illustrated in FIG. 1A, it will be clear that a prosthetic valve 100 can include any other number of leaflets 182. Adjacent leaflets 182 can be arranged together to form commissures 184 that are coupled (directly or indirectly) to respective portions of the frame 102, thereby securing at least a portion of the leaflet assembly 180 to the frame 102. The leaflets 182 can be made from, in whole or part, biological material (e.g., pericardium), bio-compatible synthetic materials, or other such materials. Further details regarding transcatheter prosthetic heart valves, including the manner in which the leaflet assemblies 180 can be coupled to the frame 102 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

The prosthetic valve 100 can further comprise at least one skirt or sealing member. An inner skirt 178 can be secured to the inner surface of the frame 102, configured to function, for example, as a sealing member to prevent or decrease perivalvular leakage. An inner skirt 178 can further function as an anchoring region for the leaflets 182 to the frame 102, and/or function to protect the leaflets 182 against damage which may be caused by contact with the frame 102, for example during valve crimping or during working cycles of the prosthetic valve 100. Additionally, or alternatively, the prosthetic valve 100 can comprise an outer skirt 176 mounted on the outer surface of the frame 102, configure to function, for example, as a sealing member retained between the frame 102 and the surrounding tissue of the native annulus against which the prosthetic valve is mounted, thereby reducing risk of paravalvular leakage (PVL) past the prosthetic valve 100.

Any of the inner skirt 178 and/or outer skirt 176 can be made of various suitable biocompatible materials, such as, but not limited to, various synthetic materials (e.g., PET) or natural tissue (e.g. pericardial tissue). In some examples, the inner skirt 178 comprises a single sheet of material that extends continuously around the inner surface of the frame 102. In some examples, the outer skirt 176 comprises a single sheet of material that extends continuously around the outer surface of the frame 102.

FIGS. 1B and 2 show the frame 102 of the prosthetic valve 100 with the other components, such as leaflets and skirts, removed. While FIG. 1B shows the frame 102 in an annular configuration, corresponding to its functional configuration. FIG. 2 shows the frame 102 in a flat configuration for purposes of illustration. In the examples illustrated in FIGS. 1B and 2, the frame 102 comprises four cell rows 136, each cell row 136 comprising a plurality of cells 130 extending circumferentially such that each cell 130 is directly coupled to two circumferentially adjacent cells 130 on both sides thereof within the same cell row 136. The term “cell”, as used herein, refers to a closed cell, having an enclosed perimeter defined by at least four struts 108.

The junctions 150 include apices 152 which are the uppermost and lowermost junctions of the frame 102, and non-apical junctions 162 disposed between the inflow end 106 and outflow end 104. The apices 152 include outflow apices 154 defined as the uppermost junctions, at least some of which can define the outflow end 104 of the valve 100, and inflow apices 160 defined as the lowermost junctions, at least some of which can define the inflow end 106 of the valve 100.

Each cell 130 can include a proximal junction 166, which is the uppermost junction of the cell, and at least one distal junction 164, which is a lowermost junction of the cell. The proximal junctions 166 of the cells 130 of the first cell row 140 are also the outflow apices 154, while the proximal junctions 166 of the cells 130 of all other cell rows, such as cell rows 142, 144 and 146, are non-apical junctions 162. The distal junctions 164 of the cells 130 of the fourth cell row 146, which is the lowermost cell row in the example illustrated in FIGS. 1B and 2, are also the inflow apices 160, while the distal junction 164 of the cells 130 of all other cell rows, such as cell rows 140, 142 and 144, are non-apical junctions 162.

Non-apical junctions 162 can also include lateral junctions 168 connecting adjacent cells 130, such as lateral junctions 168 defined between circumferentially adjacent cells 130 of the first cell row 140, the second cell row 142, and third cell row 144, of the illustrated example.

The angled struts 110 of each cell 130 can include two proximal struts 112 and two distal struts 118. Distal struts 118 of each cell 130 are diverging from a distal junction 164 upwards and sideways, or in other words, extending circumferentially away from distal junction 164 and axially towards outflow end 104. Proximal struts 112 of each cell 130 are diverging from an proximal junction 166 downwards and sideways, or in other words, extending circumferentially away from proximal junction 166 and axially towards inflow end 106.

As mentioned above, a cell 130 can include lateral junction 168, such as two lateral junction 168 at both sides thereof. In the example illustrated in FIGS. 1A and 2, cells 130 of the second cell row 142 and third cell row 144 include lateral junctions 168 at which distal struts 118 intersect with corresponding proximal struts 112, such that cells 130 are coupled to circumferentially adjacent cells 130 within cell rows 142, 144 via lateral junction 168.

Some angled struts 110 can be shared by cells 130 of two cell rows 136. For example, the distal struts 118 of cells 130 of the second cell row 142 are also the proximal struts 112 of the cells 130 of the third cell row 144, and the distal struts 118 of cells 130 of the third cell row 144 are also the proximal struts 112 of the cells 130 of the fourth cell row 146 in the illustrated example.

Each strut 108 has a length L defined between two junctions 150 on both ends thereof. For struts having non-linear shapes, the length L is defined as the linear distance between the junctions 150 on both ends. The outflow vertical strut 122 has a length L1, the proximal strut 112 has a length L2, the distal strut 118 has a length L5, and the inflow vertical strut 128 has a length L6. In the example illustrated in FIG. 2, length L2 of all proximal struts 112 of all cell 130 is equal to length L5 of all distal struts.

In some examples, cells 130 are coupled to adjacent cells 130 within the same cell row 136 via vertical struts 120. FIGS. 1A and 2 show an example of a frame 102 that includes at least two types of vertical struts 120, namely outflow vertical struts 122 defined between cells 130 of the first cell row 140, and inflow vertical struts 128 defined between cells 130 of the fourth cell row 146. The lengths L1 of the outflow vertical struts 122 and L6 of the inflow vertical struts 128 can be identical or different from one another, such as the outflow vertical struts 122 shown to have a length L1 greater than the length L6 of the inflow vertical struts 128 in the example illustrated in FIG. 2.

Vertical struts 120 can be used to interconnect the proximal struts 112 with the corresponding distal struts 118 of the same cells 130. Lateral junction 168 can include distal lateral junctions 170 and proximal lateral junctions 172 at both ends of the vertical struts 120. For example, each vertical strut 120 can extend between a distal lateral junction 170 at which it intersect with a corresponding distal strut 118, and a proximal lateral junction 170 at which it intersects with a proximal strut 112. Thus, any cell 130 that include vertical struts 120 can be defined by at least six struts: two proximal struts 112, two vertical struts 120, and at least two distal struts 118.

In some examples, at least some (e.g., three) of the outflow vertical struts 122 can be commissure support struts 124 that can define axially extending window frame portions, also termed commissure windows 126, configured to mount respective commissures 184 of the leaflet assembly 180.

Each cell 130 has a height H defined as the axial distance between its proximal junction 166 and its (one or more) distal junction/s 164, and a width W defined as the lateral or circumferential distance between its opposite lateral junctions 168, including between vertical struts 120 defining lateral junctions 170, 172 for the relevant cells 130. Since a valve expands in diameter and foreshortens during expansion, the height H of all cells will also foreshorten during the transition between a compressed and an expanded state. It is to be understood that the height H of any cell 130 referred to throughout this specification, relates to the height of the cell in the expanded state of the frame.

In the example illustrated in FIGS. 1B and 2, the cells 130 of the first cell row 140 have a height H1, the cells 130 of the second and third cell rows 142, 144 have a height H2, and the cells 130 of the fourth cell row 146 have a height H3, such that H1 is greater than H3, and H3 is greater than H2. The height H3 of the cells of the fourth cell row 146, being greater than the height H2 of the second and third cells rows 142, 144, allows the frame 102, when crimped, to assume an overall tapered shape that tapers from a larger diameter at the outflow end 104 to a narrower diameter at the inflow end 106, as further described in U.S. Pat. No. 9,393,110, which is incorporated herein by reference. As shown in FIG. 1, any of the outer skirt 176 and/or inner skirt 178 can cover the inflow portion of the frame 102, and due to the reduced diameter of this portion, such skirts need not increase the overall crimp profile of the prosthetic valve.

The exemplary frame 102 illustrated in FIGS. 1B and 2 may be representative of specific types of conventional frames known in the art, in which all cells 130 of all cell rows 136 have the same width W, resulting in an identical number of cells 130 in each cell row 136. While each cell row 136 of the illustrated example is shown to include twelve cells, it is to be understood that the number of cells in each cell row 136 can be different in other frame configurations, but will generally be identical for all cell rows 136.

A prosthetic valve 100 may be expanded against a calcified aortic annulus, requiring it to overcome the relatively increased rigidity of the calcified tissue during expansion. One of the factors known to affect the radial force exerted by the frame 102 of a valve 100 on the surrounding anatomy is the number of cells 130 in cell rows 136, wherein a greater number of cells 130 (i.e., a higher cell density) will result in a greater radial force during expansion. Thus, conventional valve frames 102 may be provided with a relatively large number of cells 130 to increase the radial force to overcome the resistance of the calcified annular pathologies.

Some prosthetic valves may have an overall axial length, in their expanded state, that can place the upper or first cell row (140) at the level of the coronary ostia. For example, such valves (100) can be designed to have their outflow apices (154) contacting or being placed in the vicinity of the sinuses or the Sinotubular Junction (STJ) when expanded at the site of implantation. In some instances, a patient may require implantation of a coronary stent of other procedure that requires access to a coronary artery after prosthetic valve implantation. For such instances, a physician may need to access the coronary artery through the opening defined by a cell 130 of the first cell row 140 facing the coronary ostium. While the cells 130 of the first cell row 140 of the prosthetic valve 100 illustrated, for example, in FIGS. 1A-2, may have a height H1 which is greater than the heights H2 and H3 of other cell rows 142, 144, 146, their width W is identical to the width of all other cell 130. Since the number of cells can be chosen to be relatively large to provide sufficient radial force during expansion against the native annulus, this may result in a cell width W that can compromise the ability for future access into the coronary arteries or perfusion through the frame 102 to the coronary arteries during the diastolic phase of the cardiac cycle.

Taking advantage of the fact that increased radial force during valve expansion is required primarily at the region of the native annulus, corresponding to the lower portion (i.e., distal or inflow portion) of the frame, an upper cell row of frame configurations disclosed herein can include wider cells than those of the other cell rows. The width of the wider cells in the first cell row can be configured to be larger than the outer diameter of a selected coronary catheter (e.g., a 6 Fr coronary catheter).

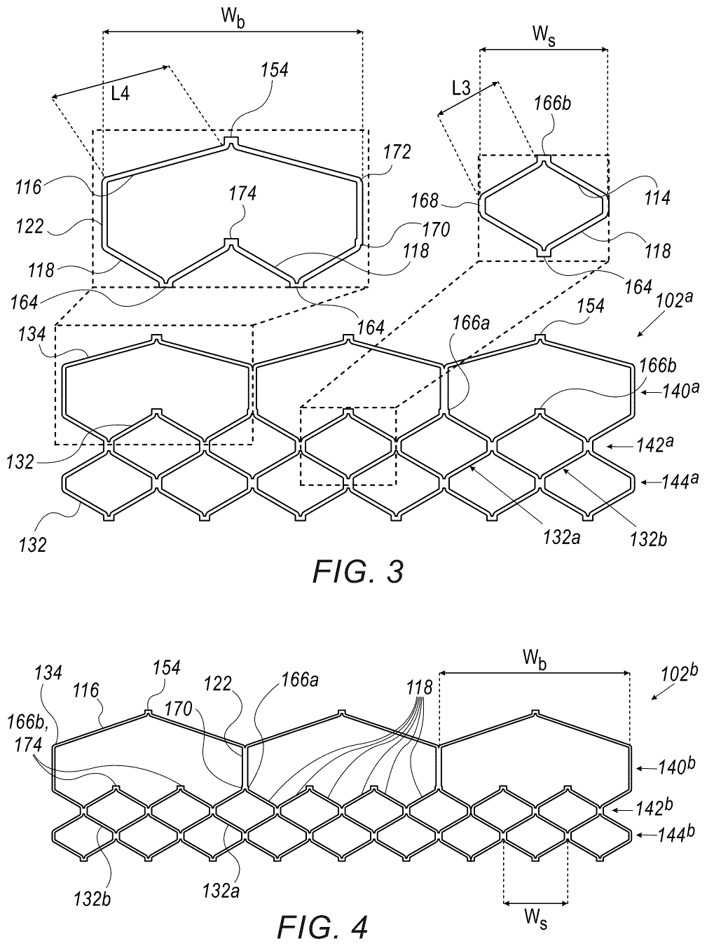

FIGS. 3-16 show various frame configurations disclosed herein, comprising wide cells in the first cell row, having a width Wb which is a multiple of the width Ws of narrow cells comprised in the second and third cell rows. The width Wb being a multiple of Ws means that Ws is multiplied by an integer, preferably 2 or greater, to derive Wb. The frames (or portions thereof) are shown throughout FIGS. 3-16 in a flattened state and without soft components (such as leaflets and skirts) for clarity. Moreover, while frames are illustrated throughout FIGS. 3-16 showing a first cell row, a second cell row, and a third cell row, it is to be understood that this is shown by way of illustration and not limitation, and that the same frames can include additional rows of cells. For example, any of the frames described below in conjunction with FIGS. 3-16 can include a fourth cell row with cells that can be similar or different than those of the fourth cell row 146 illustrated in FIG. 2, as well as any number of additional rows of cells.

According to some examples, the cells 130 include narrow cells 132 and wide cells 134, wherein the width Wb of the wide cells 134 is greater than the width Ws of the narrow cells 132, and is a multiple of Ws. For example, the width Wb of wide cells 134 can be two times, three times, four times, five times or six times as great as the width Ws of narrow cells 132.

It is to be understood that various narrow cells 132 can have the same width Ws but a different overall size, such as by having different heights H. For example, all cells 130 of the frame 102 illustrated in FIG. 2 can be regarded as narrow cells 132 having identical widths Ws, yet the narrow cells 132 of the first cell row 140 have a greater height H1 than the height H2 of the narrow cells 132 of the second and third cells rows 142, 144.

In some examples, the second cell row and third cell row include only narrow cells 132, while the first cell row 140 comprises wide cells 134, with or without narrow cells 132. All of the narrow cells 132 of any of the second cell row 142 and third cell row 144 can have an identical size, meaning that all narrow cells 132 of the second cell row 142 and/or the third cell row 144 have not only identical widths Ws, but also identical heights H2.

The wide cells 134 can reduce the amount of metal in the outflow end portion of the frame 102, which, because it aligns with the bulk volume of the leaflets when the prosthetic valve is radially crimped, allows for a smaller crimping diameter of the prosthetic valve. The wide cells 134 ensure adequate blood flow through the prosthetic valve to the coronary ostia, once the prosthetic valve has been implanted, as well as allowing re-access devices (e.g., coronary catheters) to pass through the wide cells to access the coronary vessels. For example, each wide cell 134 can be configured to be twice as wide as the outer diameter of a selected coronary catheter (e.g., a 6 Fr coronary catheter).

The narrow cells 132 (which are narrower and generally smaller when compared to wide cells 134), such as the cells in the second and third cell rows in the exemplary frames illustrated throughout FIGS. 3-9, can have a relatively stronger structural strength than wide cells 134. Accordingly, the frame can be positioned within the native annulus such that narrow cells 132 in lower cell rows (such as cell rows 142, 144 and 146 when present) bear a greater amount of the radial force applied by the native annulus than the wide cells 134.

In some examples, the first cell row comprises wide cells 134 having a width Wb at least twice as great as the width Ws of any of the narrow cells 132 of the second cell row and/or the third cell row. FIG. 3 shows an example of a frame 102^{a} of a prosthetic valve 100^{a}, which is similar in structure and function to frame 102 of prosthetic valve 100 disclosed above with respect to FIGS. 1A-2, except that the frame 102^{a} comprises a first cell row 140^{a} with three wide cells 134, and a second and third cell rows 142^{a}, 144^{a}, each having six narrow cells 132, resulting in the width Wb of the wide cells 134 of frame 102^{a} being twice as great as the width Ws of the narrow cells 132.

Various exemplary implementations for prosthetic valve 100 and components thereof, can be referred to, throughout the specification, with superscripts, for ease of explanation of features that refer to such exemplary implementations. It is to be understood, however, that any reference to structural or functional features of any device or component, without a superscript, refers to these features being commonly shared by all specific exemplary implementations that can be also indicated by superscripts. In contrast, features emphasized with respect to an exemplary implementation of any device or component, referred to with a superscript, may be optionally shared by some but not necessarily all other exemplary implementations.

In some examples, wide cells 134 of any of the frame configurations disclosed herein with respect to FIGS. 3-16 can include outflow vertical struts 122 extending on both sides of each wide cell 134 between the distal struts 118 and the proximal struts 112, such that each wide cell 134 of the first cell row is coupled to adjacent cells 130 (e.g., wide cells 134) on both sides via the outflow vertical struts 122. In some examples, the wide cells of the first cell row can be devoid of vertical struts (examples not shown), such that each wide cell can be coupled to two adjacent wide cells on both sides thereof via corresponding lateral junctions.

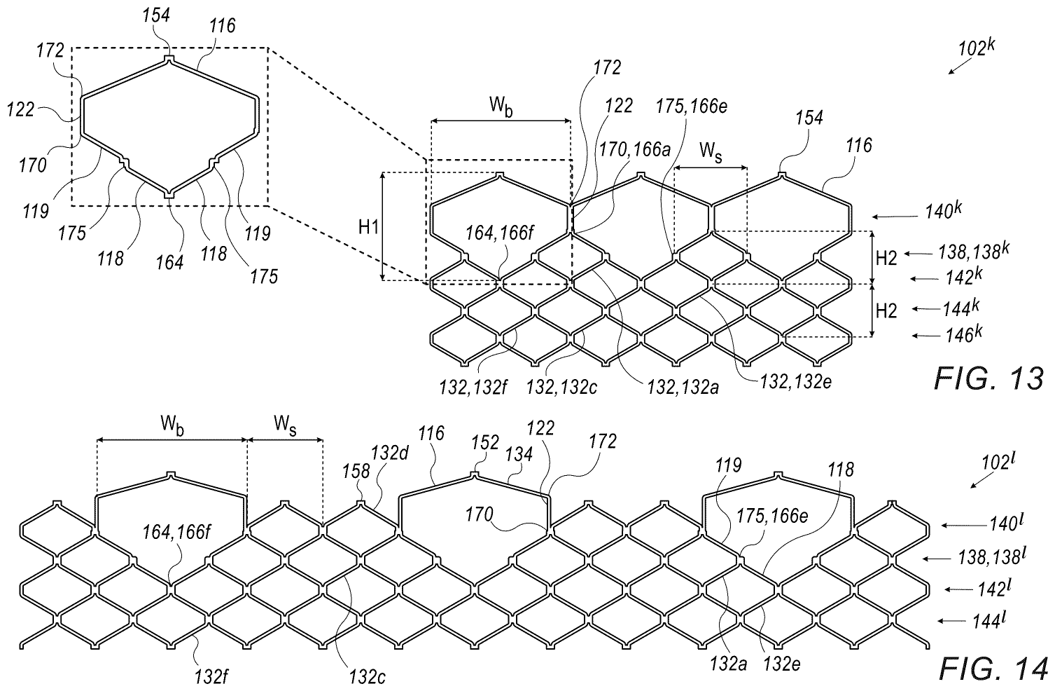

Some of the illustrated examples, such as the example illustrated in FIG. 3, show narrow cells 132 having a diamond shape, and wide cells 134 are shown to have a substantially chevron shape (or a diamond shape for the frame configurations illustrated in FIGS. 13-14). However, in other examples, any of the cells 130 can be hexagonal, octagonal, triangular, diamond-shaped, diamond shaped having tapered end portions, etc.

While not illustrated throughout FIGS. 3-16 for simplicity, it is to be understood that for frame configurations that do include outflow vertical struts 122 comprised in the wide cells 134, at least some of the outflow vertical struts 122 can be commissure support struts 124 that optionally define commissure windows 126. Commissure windows 126 can be designed to allow tabs of leaflets 182 to pass therethrough in order to form commissures 184. It is to be understood that other commissure support structures may be available, such as vertical struts that do not necessarily include commissure windows, for example designed to support tabs of leaflets that can be wrapped therearound and sutured or otherwise coupled thereto, to form commissures 184.

The proximal struts 112 of frames that include both narrow cells 132 and wide cells 134, can comprise first-length proximal struts 114 having a length L3 and second-length proximal struts 116 having a length L4, wherein the second-length proximal struts 116 can be longer than the first-length proximal struts 114 (i.e., L4>L3), and in some cases may have a length L4 which is generally a multiple of the length L3. Length L3 may be generally equal to length L5 of the distal struts 118. In some examples, the ratio between the lengths L4 and L3 can be equal to or greater than the ratio between W1 and W2.

Each wide cell 134 of the exemplary frame 102^{a} illustrated in FIG. 3 includes two second-length proximal struts 116 diverging from an outflow apex 154 towards two corresponding outflow vertical struts 122, and four distal struts 118 arranged in a zig-zagged pattern, which are also the first-length proximal struts 114 of narrow cells 132 of the second cell row 142^{a}. In this manner, each such wide cell 134 has two distal junctions 164, which are also lateral junctions 168 of narrow cells 132 of the second cell row 142^{a}, as well as proximal junctions 166 of narrow cells 132 of the third cell row 144^{a}.

In some examples, a wide cell 134 can further comprise at least one free junction 174, which is another type of a non-apical junction 162, disposed between the downstream 164 and upstream 166 junctions of the wide cell 134, and between lateral junctions 168 (including between outflow vertical struts 122) of the wide cell 134. The term “free junction”, as used herein, refers to a junction that is not attached (directly attached) to any other struts, except for two distal struts 118 diverging therefrom (and defining the free junction 174 at the intersection therebetween). A free junction 174 is distal to the outflow apex 154 and proximal to the one or more distal junction/s 164 of the wide cell 134 it is comprised in.

Narrow cells 132 can include several types of cells, such as narrow cells 132a and 132b, which are identical in shape and size, but may differ by the adjacent cells they are attached to. A narrow cell 132a of any second cell row or an intermediary cell row (which will be described further below) of the various examples of the current specification, will have its proximal junction 166a converging with a lateral junction of a wide cell 134 of the first cell row. For example, each proximal junctions 166a of a narrow cell 132a can converge with a distal lateral junction 170 of at least one wide cell 134. In some cases, the proximal junctions 166a are disposed between the wide cells 134, for example converging with the distal lateral junctions 170 defined between two wide cells 134 directly coupled to each other at the first cell row. In contrast, each narrow cell 132b of any second cell row of the various examples of the current specification, has its first-length proximal struts 114 extending towards the center of a corresponding wide cell 134, such that its free-ended proximal junction 166b is also a free junction 174 of the corresponding wide cell 134.

Any junction termed to “converge” with another junction, throughout the current specification, means that both junctions are one and the same. For example, a proximal junction of a first cell, termed to converge with a lateral junction of a second cell, means that the proximal junction of the first cell is also the lateral junction of the second cell.

In the example illustrated in FIG. 3, half of the narrow cells 132 (i.e., three) of the second cell row 142^{a} are narrow cells 132a, having their proximal junctions 166a disposed between wide cells 134, and the other half are narrow cells 132b, defining a total of three corresponding free junctions 174.

In some examples, as in the example illustrated in FIG. 3, each free junction 174 of a wide cell 134 (which is also the free-ended proximal junction 166b of the corresponding narrow cell 132b) is vertically aligned with the outflow apex 154 of the same wide cell 134.

The term “free-ended proximal junction” refers to an proximal junction, such as proximal junction 166b, from which only two proximal struts 112 diverge (downwards and sideways therefrom), without any other struts, such as any distal struts (118) or vertical struts (120), being connected thereto. In other words, a free-ended proximal junction 166b of any cell 130b, for example, will not serve also as a distal junction (164) or a lateral junction (168) of any other cells. An upper free-ended junction 150 of any cell 130, defined in the same manner, will be an outflow apex 154 if the frame does not include any other junction or strut disposed vertically above it, and will be a free-ended proximal junction 166b if the frame includes a strut (108) or any other junction (such as an outflow apex 154 in the example of FIG. 3) disposed vertically above it.

The example illustrated in FIG. 3 shows three wide cells 134 in the first cell row 140^{a}, wherein each wide cell 134 comprises an outflow apex 154 aligned with its free junction 174 (or otherwise stated, aligned with the free-ended proximal junction 166b of a corresponding narrow cell 132b of the second cell row 142^{a}). Any two junctions are termed to be “vertically aligned” if an imaginary line passing through both junctions, or a projection thereof on an annular plane defined around central axis 10, is parallel to central axis 10 of the prosthetic valve. In contrast, any two junctions are termed to be “laterally aligned” if they are equally distanced from the outflow end 104 and/or from the inflow end 106 of the prosthetic valve 100. For example, all of the outflow apices 154 shown in any of the frames 102 illustrated in FIG. 2 or 3 are laterally aligned. Similarly, all distal lateral junctions 170 at the lower end of the outflow vertical struts 122 are laterally aligned.

It is to be understood that each cell 130 of the first cell row, according to any of the examples described herein in conjunction with FIGS. 3-16, includes an outflow apex 154, and that all cells 130 of any first cell row according to any of the examples described herein in conjunction with FIGS. 3-16 are directly coupled to adjacent cells 130 of the first cell row, for example via shared vertical struts 120 and/or shared lateral junctions 168.

In some examples, all narrow cells 132 of any second cell row disclosed herein, including any of the second cell rows described in conjunction with FIGS. 3-16, are directly coupled to adjacent narrow cells 132 of the second cell row, for example via shared lateral junctions 168.

In some examples, all narrow cells 132 of any third cell row disclosed herein, including any of the third cell rows described in conjunction with FIGS. 3-16, are directly coupled to adjacent narrow cells 132 of the second cell row, for example via shared lateral junctions 168.

In some examples, all cells 130 of the first cell row are wide cells 134, such that each wide cell 134 is directly coupled to adjacent wide cells 134 on both sides thereof, such as shown for frame 102^{a} illustrated in FIG. 3, as well as for frame 102^{b}, 102^{c}, 102^{d}, 102^{e}, 102^{f}, 102^{g}, 102^{k}, 100^{m} and 100ⁿ as will be further described hereinbelow and illustrated in FIGS. 4, 5, 6A-D, 7, 8, 9, 13, 15 and 16, respectively.

In some examples, all wide cells 134 of any first cell row disclosed herein, including any of the second cell rows described in conjunction with FIGS. 3-146, have the same width Wb and the same height H1, such that all outflow apices 154 of the wide cells 134 of the first cell row are laterally aligned with each other, all distal junctions 164 of the wide cells 134 of the first cell row are laterally aligned with each other, and all of the free junctions 174 are laterally aligned with each other. If wide cells 134 include outflow vertical struts 122, all distal lateral junctions 170 of the outflow vertical struts 122 are laterally aligned with each other, and all proximal lateral junctions 172 of the outflow vertical struts 122 are laterally aligned with each other.

In some examples, all narrow cells 132 of any second cell row disclosed herein, including any of the second cell rows described in conjunction with FIGS. 3-14, have the same width Ws and the same height H2, such that all proximal junctions 166 of the narrow cells 132 of the second cell row are laterally aligned with each other, all lateral junctions 168 of the narrow cells 132 of the second cell row are laterally aligned with each other, and all distal junctions 164 of the narrow cells 132 of the second cell row are laterally aligned with each other.

In some examples, all narrow cells 132 of any third cell row disclosed herein, including any of the third cell rows described in conjunction with FIGS. 3-16, have the same width Ws and the same height H2, such that all proximal junctions 166 of the narrow cells 132 of the third cell row are laterally aligned with each other, all lateral junctions 168 of the narrow cells 132 of the third cell row are laterally aligned with each other, and all distal junctions 164 of the narrow cells 132 of the third cell row are laterally aligned with each other.

In some examples, as in the example illustrated in FIG. 3 or any of the examples illustrated in FIG. 4-16, all free junctions 174 of the wide cells 134 are laterally aligned with the distal lateral junctions 170 of outflow vertical struts 122.

In some examples, the first cell row comprises wide cells 134 having a width Wb at least three times as great as the width Ws of any of the narrow cells 132 of the second cell row and/or the third cell row. FIG. 4 shows an example of a frame 102^{b} of a prosthetic valve 100^{b}, which is similar in structure and function to frame 102^{a} of prosthetic valve 100^{a} disclosed above with respect to FIG. 3, including a first cell row 140^{b} having three wide cells 134, but unlike frame 102^{a}, each of the second and third cell rows 142^{b}, 144^{b} of frame 102^{b} comprises nine narrow cells 132, resulting in the width Wb of the wide cells 134 of frame 102^{b} being three times as great as the width Ws of the narrow cells 132.

Each wide cell 134 of the exemplary frame 102^{b} illustrated in FIG. 4 includes two second-length proximal struts 116 diverging from an outflow apex 154 towards two corresponding outflow vertical struts 122, and six distal struts 118 arranged in a zig-zagged pattern, which are also the first-length proximal struts 114 of narrow cells 132 of the second cell row 142^{b}. In this manner, each such wide cell 134 has three distal junctions 164, which are also lateral junctions 168 of narrow cells 132 of the second cell row 142^{b}, as well as proximal junctions 166 of narrow cells 132 of the third cell row 144^{b}.

The second cell row 142^{b} includes three narrow cells 132a with proximal junctions 166a disposed between the wide cells 134 of the first cell row 140^{b}, and six narrow cells 132b with free-ended proximal junction 166b. Each narrow cell 132b of the second cell row 142^{b} is directly connected to another narrow cell 132b via a shared lateral junction 168 on one side, and to a narrow cell 132a via a shared lateral junction 168 on the opposite side. Thus, each wide cell 134 of the first cell row 140^{b} includes two free junctions 174, which are the free-ended proximal junctions 166b of a respective couple of narrow cells 132b directly connected to each other. In this manner, none of the free junctions 174 is vertically aligned with the outflow apex 154 of the same wide cell 134, but rather the outflow apex 154 is vertically aligned with the lateral junction 168 connecting the two narrow cells 132b that define the free junctions 174 of the same wide cell 134. In other words, the outflow apex 154 is vertically aligned with its middle-most distal junction 164 (i.e., the central of all three distal junctions 164).

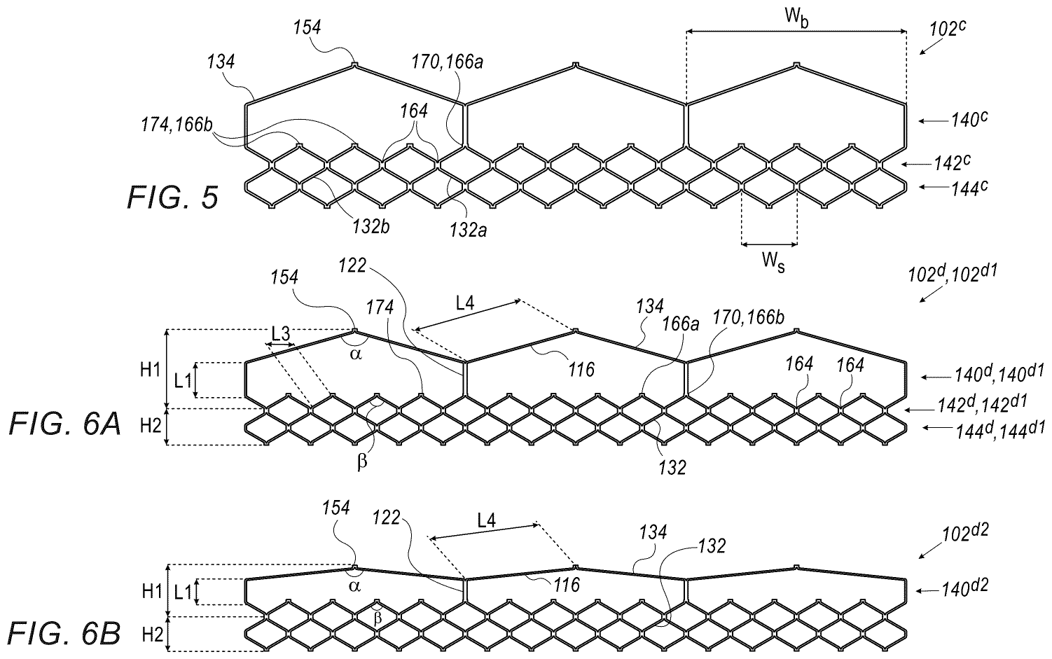

FIG. 5 shows an example of a frame 102^{c} of a prosthetic valve 100^{c}, which is similar in structure and function to frames 102^{a} or 102^{b} disclosed above with respect to FIG. 3 or 4, respectively, including a first cell row 140^{c} having three wide cells 134, but unlike frames 102^{a} or 102^{b}, each of the second and third cell rows 142^{c}, 144^{c} of frame 102^{c} comprises twelve narrow cells 132, resulting in the width Wb of the wide cells 134 of frame 102^{b} being four times as great as the width Ws of the narrow cells 132.

Each wide cell 134 of the exemplary frame 102^{c} illustrated in FIG. 5 includes two second-length proximal struts 116 diverging from an outflow apex 154 towards two corresponding outflow vertical struts 122, and eight distal struts 118 arranged in a zig-zagged pattern, which are also the first-length proximal struts 114 of narrow cells 132 of the second cell row 142^{c}. In this manner, each such wide cell 134 has four distal junctions 164, which are also lateral junctions 168 of narrow cells 132 of the second cell row 142^{c}, as well as proximal junctions 166 of narrow cells 132 of the third cell row 144^{c}.

The second cell row 142^{c} includes three narrow cells 132a with proximal junctions 166a disposed between the wide cells 134 of the first cell row 140^{c}, and nine narrow cells 132b with free-ended proximal junction 166b. Each narrow cell 132b of the second cell row 142^{c} is directly connected at least one other narrow cell 132b, wherein three of the narrow cells 132b are each directly connected to two other narrow cells 132b via shared lateral junction 168 on both sides. Each of the six other narrow cells 132b is connected, in turn, to another narrow cell 132b via a shared lateral junction 168 on one side, and to a narrow cell 132a via a shared lateral junction 168 on the opposite side. Thus, each wide cell 134 of the first cell row 140^{c} includes three free junctions 174, which are the free-ended proximal junctions 166b of a respective triplet of narrow cells 132b. In this manner, the middle-most free junction 174 of the three free junctions of a wide cell 134 is vertically aligned with the outflow apex 154 of the same wide cell 134.

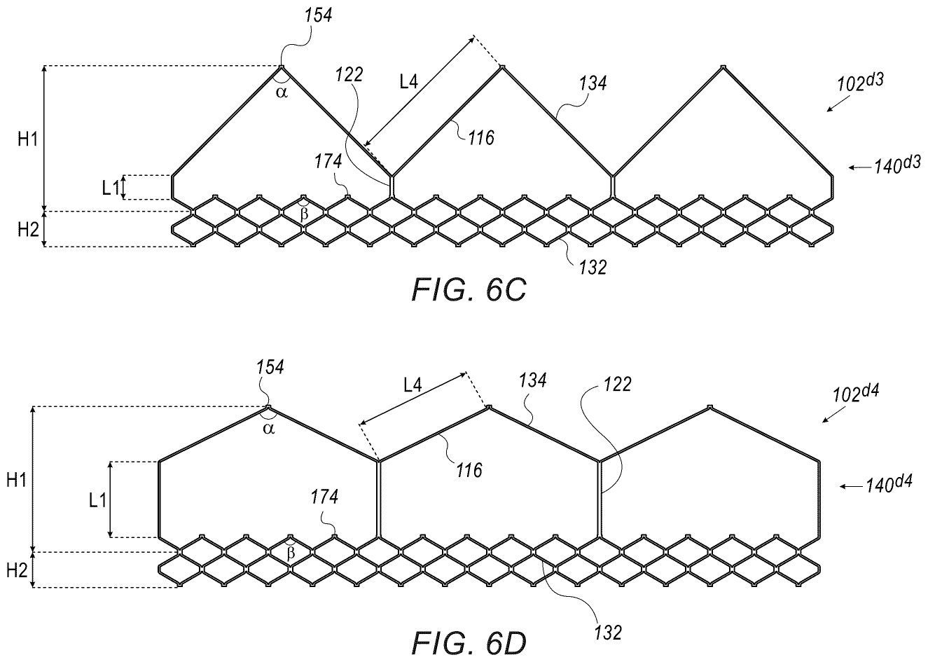

FIGS. 6A-D show four examples of a frame 102^{d} of a prosthetic valve 100^{d}, which is similar in structure and function to frames 102^{a}, 102^{b} or 102^{c} disclosed above with respect to FIG. 3, 4 or 5, respectively, including a first cell row 140^{d} having three wide cells 134, wherein unlike frames 102^{a}, 102^{b} or 102^{c}, each of the second and third cell rows 142^{d}, 144^{d} of frame 102^{d} comprises fifteen narrow cells 132, resulting in the width Wb of the wide cells 134 of frame 102^{d} being five times as great as the width Ws of the narrow cells 132.

Each wide cell 134 of the any exemplary frame 102^{d} illustrated in FIGS. 6A-D includes two second-length proximal struts 116 diverging from an outflow apex 154 towards two corresponding outflow vertical struts 122, and ten distal struts 118 arranged in a zig-zagged pattern, which are also the first-length proximal struts 114 of narrow cells 132 of the second cell row 142^{d}. In this manner, each such wide cell 134 has five distal junctions 164, which are also lateral junctions 168 of narrow cells 132 of the second cell row 142^{d}, as well as proximal junctions 166 of narrow cells 132 of the third cell row 144^{d}.

The second cell row 142^{d} includes three narrow cells 132a with proximal junctions 166a disposed between the wide cells 134 of the first cell row 140^{d}, and twelve narrow cells 132b with free-ended proximal junction 166b. Each narrow cell 132b of the second cell row 142^{d} is directly connected at least one other narrow cell 132b, wherein six of the narrow cells 132b are each directly connected to two other narrow cells 132b via shared lateral junction 168 on both sides. Each of the six other narrow cells 132b is connected, in turn, to another narrow cell 132b via a shared lateral junction 168 on one side, and to a narrow cell 132a via a shared lateral junctions 168 on the opposite side. Thus, each wide cell 134 of the first cell row 140^{d} includes four free junctions 174, which are the free-ended proximal junctions 166b of a respective quadruplet of narrow cells 132b. In this manner, none of the free junctions 174 of a wide cell 134 is vertically aligned with its outflow apex 154, but rather the outflow apex 154 is aligned with the lateral junction 168 connecting the two middle-most narrow cells 132b that define the free junctions 174 of the same wide cell 134. In other words, the outflow apex 154 is vertically aligned with its middle-most distal junction 164 (i.e., the central of all five distal junctions 164).

The second-length proximal struts 116 can define a first angle α therebetween, and the first-length proximal struts 114 can define a second angle β therebetween. Any of the angles α and/or β is oriented towards inflow end 106 as shown in FIG. 6A for example. FIGS. 6A, 6B, 6C and 6D show frame configuration 102^{d1}, 102^{d2}, 102^{d3} and 102^{d4}, respectively, which are four examples of the frame 102^{d} described hereinabove, with the distinction that each shows differently shaped wide cells 134. FIG. 6A shows an example of a frame 102^{d1} in which the first angle α is substantially equal to the second angle β. The term “substantially equal”, as used herein, refers to one value (such as the angle α) being in the range of ±10% of the other value (such as the angle β).

In all of the examples illustrated in FIGS. 6A-D, the wide cells 134 include outflow vertical struts having the length L1. For configurations in which some of the outflow vertical struts 122 are commissure support struts 124 that include commissure windows 126, the length L1 can be dictated by the minimal length required for the commissure windows 126 that need to accommodate commissures 184 passing therethrough. As indicated above, the length L4 an second-length proximal strut 116 is greater than the length L3 of a first-length proximal strut 114, and the height H1 of a wide cell 134 of the first cell row 140 is greater than the height H2 of a narrow cell 132 of any of the second or third cell rows 142, 144. In the example of frame 102^{d1} illustrated in FIG. 6A, the length L1 is shown to be greater than the length L3, and more specifically, the length L1 can be in the range between L3 and 2*L3 (that it, greater than L3 but not greater than twice the length L3).

FIG. 6B shows an example of a frame 102^{d2} in which the first angle α is greater than the second angle β. The length L1 shown is FIG. 6B is shorter than the length L1 shown in FIG. 6A. For example, the length L1, as illustrated in FIG. 6B, can be substantially equal to the length L4. Both of these feature result in an overall height H1 shown in FIG. 6B to be shorter than the height H1 shown in FIG. 6A.

FIG. 6C shows an example of a frame 102^{d3} in which the length L1 can be similar to that shown in FIG. 6B, but with the first angle α being sharper than the second angle β. The shorter the length L1 is, and the sharper the first angle α is, the longer the length L4 will be. For example, the length L4 illustrated in FIG. 6C is shown to be considerably greater than that shown in any of the FIG. 6A or 6B, resulting also in a height H1 greater than that shown in any of the FIG. 6A or 6B.

FIG. 6D shows an example of a frame 102^{d4} in which the height H1 can be similar to that shown in FIG. 6C, and in which the first angle α is substantially equal to the second angle β, in a similar manner to that described for these angles with respect to FIG. 6A. These features will result in longer L4 lengths of outflow vertical struts 122, shown in FIG. 6D to be considerably greater than L4 of any of FIGS. 6A-C.

While the width Wb of all wide cells 134 shown in FIG. 6A-D is identical, their height H1 can vary as a function of various factors influencing the cell's shape, including, but not limited to: the length L1 of the outflow vertical struts 122, the length L4 of the second-length proximal struts 116, the first angle α defined between the second-length proximal struts 116, and any combination thereof.

In general, smaller height H1 of the wide cells 134, such as the configuration shown in FIG. 6B, will result in a smaller height of the whole frame 102 (between the inflow end 106 and the outflow end 104), which can advantageously, for some frame configurations, can position the outflow apices 154 closer to the coronary sinuses and/or below the STJ. However, such configurations may also require additional means for initiating crimping and prevent buckling of their relatively flattened elongated struts. In contrast, a significantly greater height H1, such as illustrated in FIG. 6C or 6D, may enable easier crimping of the valve, but may result in the valve being too long and protruding too far into the ascending aorta, optionally with the inflow portions of the valve that include the base of the leaflet assembly 180 and outer or inner skirts 176, 178 covering the coronary ostia in a manner that disturbs the blood flow into the coronaries, and may prevent access of other devices (such as coronary catheters) into the coronary arteries, if such procedures are required after prosthetic valve implantation.

Thus, the shape and dimensions of wide cells 134 will be set according to required design choices, accounting for the factors described above. It is to be understood that while shown for frame configuration 102^{d} throughout FIG. 6A-D, this is meant only to illustrate example of the various factors that can influence the shape and dimensions of wide cells 134, and that any of the design options described above in conjunction with FIG. 6A-D are similarly applicable to any of the other wide cells of the frame configuration described in conjunctions with FIGS. 3-5 and 7-14. Moreover, while illustrated and described with respect to FIGS. 6A-D for wide cells 134 that include outflow vertical struts 122, it is to be understood that similar shape and size manipulation can be implemented for wide cells that do not include vertical struts (being connected to each other, for example, via mutual lateral junctions), in which case manipulation of lengths L1 is irrelevant, yet any of the lengths L4 and the first angle α can be manipulated in the same manner described above.

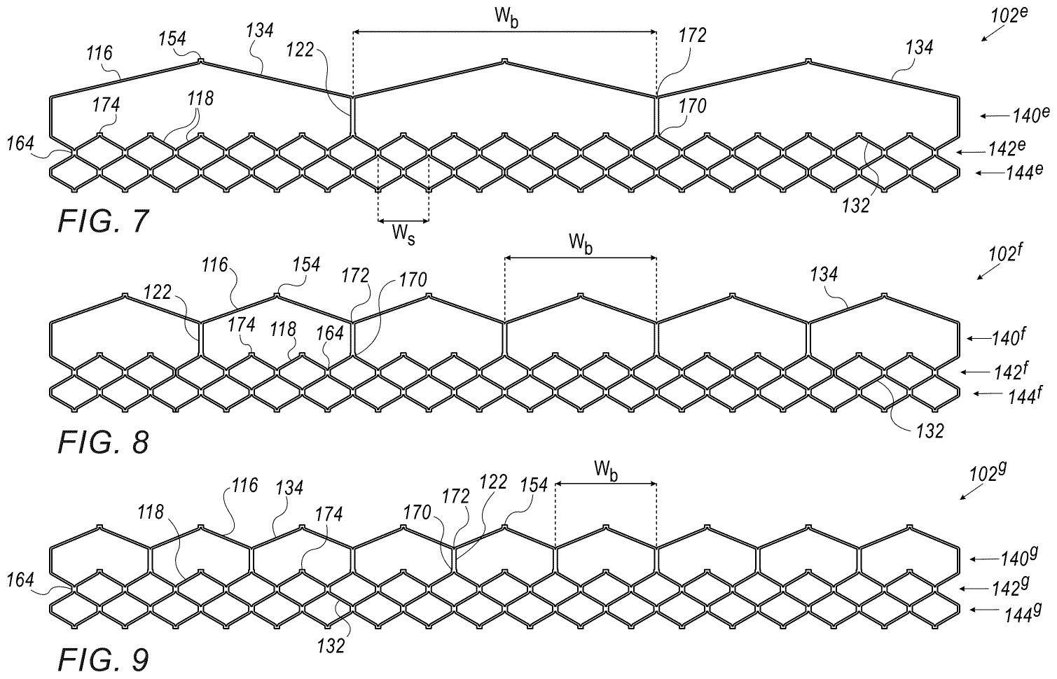

FIG. 7 shows an example of a frame 102^{e} of a prosthetic valve 100^{e}, which is similar in structure and function to frames 102^{a}, 102^{b}, 102^{c} or 102^{d} disclosed above with respect to FIG. 3, 4, 5 or 6A-D, respectively, including a first cell row 140^{d} having three wide cells 134, wherein unlike frames 102^{a}, 102^{b}, 102^{c} or 102^{d}, each of the second and third cell rows 142^{e}, 144^{e} of frame 102^{e} comprises eighteen narrow cells 132, resulting in the width Wb of the wide cells 134 of frame 102^{e} being six times as great as the width Ws of the narrow cells 132.

Each wide cell 134 of the exemplary frame 102^{e} illustrated in FIG. 7 includes two second-length proximal struts 116 diverging from an outflow apex 154 towards two corresponding outflow vertical struts 122, and twelve distal struts 118 arranged in a zig-zagged pattern, which are also the first-length proximal struts 114 of narrow cells 132 of the second cell row 142^{e}. In this manner, each such wide cell 134 has six distal junctions 164, which are also lateral junctions 168 of narrow cells 132 of the second cell row 142^{e}, as well as proximal junctions 166 of narrow cells 132 of the third cell row 144^{e}.

The second cell row 142^{e} includes three narrow cells 132a with proximal junctions 166a disposed between the wide cells 134 of the first cell row 140^{e}, and fifteen narrow cells 132b with free-ended proximal junction 166b. Each narrow cell 132b of the second cell row 142^{e} is directly connected at least one other narrow cell 132b, wherein nine of the narrow cells 132b are each directly connected to two other narrow cells 132b via shared lateral junctions 168 on both sides. Each of the six other narrow cells 132b is connected, in turn, to another narrow cell 132b via a shared lateral junction 168 on one side, and to a narrow cell 132a via a shared lateral junction 168 on the opposite side. Thus, each wide cell 134 of the first cell row 140^{e} includes five free junctions 174, which are the free-ended proximal junctions 166b of a respective quintuplet of narrow cells 132b. In this manner, the middle-most free junction 174 of the five free junctions of a wide cell 134 is vertically aligned with the outflow apex 154 of the same wide cell 134.

FIG. 8 shows an example of a frame 102^{f} of a prosthetic valve 100^{f}, which is similar in structure and function to frame 102^{e} described above with respect to FIG. 7, including second and third cell rows 142^{f}, 144^{f} comprising eighteen narrow cells 132a, wherein unlike frame 102^{e}, the first cell row 140^{f} of frame 102^{f} comprises six wide cells 134 instead of three, resulting in the width Wb of the wide cells 134 of frame 102^{f} being three times as great as the width Ws of the narrow cells 132.

Each wide cell 134 of the exemplary frame 102^{f} illustrated in FIG. 8 includes two second-length proximal struts 116 diverging from an outflow apex 154 towards two corresponding outflow vertical struts 122, and six distal struts 118 arranged in a zig-zagged pattern, which are also the first-length proximal struts 114 of narrow cells 132 of the second cell row 142^{f}. In this manner, each such wide cell 134 has three distal junctions 164, which are also lateral junctions 168 of narrow cells 132 of the second cell row 142^{f}, as well as proximal junctions 166 of narrow cells 132 of the third cell row 144^{f}.

The second cell row 142^{f} includes six narrow cells 132a with proximal junctions 166a disposed between the wide cells 134 of the first cell row 140^{f}, and twelve narrow cells 132b with free-ended proximal junction 166b. Each narrow cell 132b of the second cell row 142^{f} is directly connected to another narrow cell 132b via a shared lateral junction 168 on one side, and to a narrow cell 132a via a shared lateral junction 168 on the opposite side. Thus, each wide cell 134 of the first cell row 140^{f} includes two free junctions 174, which are the free-ended proximal junctions 166b of a respective couple of narrow cells 132b directly connected to each other. In this manner, none of the free junctions 174 is vertically aligned with the outflow apex 154 of the same wide cell 134, but rather the outflow apex 154 is aligned with the lateral junction 168 connecting the two narrow cells 132b that define the free junctions 174 of the same wide cell 134.

FIG. 9 shows an example of a frame 102^{g} of a prosthetic valve 100^{g}, which is similar in structure and function to frames 102^{e} or 102^{f} described above with respect to FIG. 7 or 8, respectively, including second and third cell rows 142^{g}, 144^{g} comprising eighteen narrow cells 132, wherein unlike frame 102^{e} or 102^{f}, the first cell row 140^{g} of frame 102^{g} comprises nine wide cells 134, resulting in the width Wb of the wide cells 134 of frame 102^{g} being twice as great as the width Ws of the narrow cells 132.

Each wide cell 134 of the exemplary frame 102^{g} illustrated in FIG. 9 includes two second-length proximal struts 116 diverging from an outflow apex 154 towards two corresponding outflow vertical struts 122, and four distal struts 118 arranged in a zig-zagged pattern, which are also the first-length proximal struts 114 of narrow cells 132 of the second cell row 142^{g}. The second cell row 142^{g} includes nine narrow cells 132a with proximal junctions 166a disposed between the wide cells 134 of the first cell row 140^{g}, and nine narrow cells 132b with free-ended proximal junction 166b. Each narrow cell 132b of the second cell row 142^{g} is directly connected to two narrow cell 132a via shared lateral junctions 168 on both sides thereof. Thus, each wide cell 134 of the first cell row 140^{g} includes a single free junction 174, which is the free-ended proximal junction 166b of the respective narrow cell 132b. In this manner, each free junction 174 is vertically aligned with the outflow apex 154 of the same wide cell 134.

In some examples, a frame of a prosthetic valve includes a first cell row with a plurality of wide cells 134 and a plurality of narrow cells 132, while the second and third cell rows include only narrow cells 132, wherein each width Wb of each wide cell 134 is at least twice as great as the width Ws of a narrow cells 132. Each cell 130 of the first cell row is directly coupled to two other cells 130, which can be either wide cells 134 or narrow cells 132, on both of its sides. In such examples, the outflow apices 154 include a plurality of first outflow apices 156 defined by the wide cells 134, and a plurality of second outflow apices 158 defined by the narrow cells 132 of the first cell row, wherein the first outflow apices 156 are higher (e.g., more proximal) than second outflow apices.

In some examples, narrow cells 132 can include several types of cells, including narrow cells 132a and 132b as described above, as well as narrow cells 132c and 132d, all of which are identical in shape and size, but may differ by the surrounding cells they are attached to. A narrow cell 132d of any first cell row of the various examples of the current specification, defines a free-ended proximal junction which is a second outflow apex 158, with two first-length proximal struts 114 diverging from the second outflow apex 158, without any other struts or junctions positioned above the first-length proximal struts 114 of any narrow cell 132d. A narrow cell 132c of any second or third cell row of the various examples of the current specification, has an proximal junction 166c which converges with a lateral junction 168 disposed between two narrow cells 132 of an upper cell row, such as narrow cells 132d of the first cell row.

In some examples, the width Wb of the wide cells 134 is exactly twice as great as the width Ws of the narrow cells 132d of the first cell row.

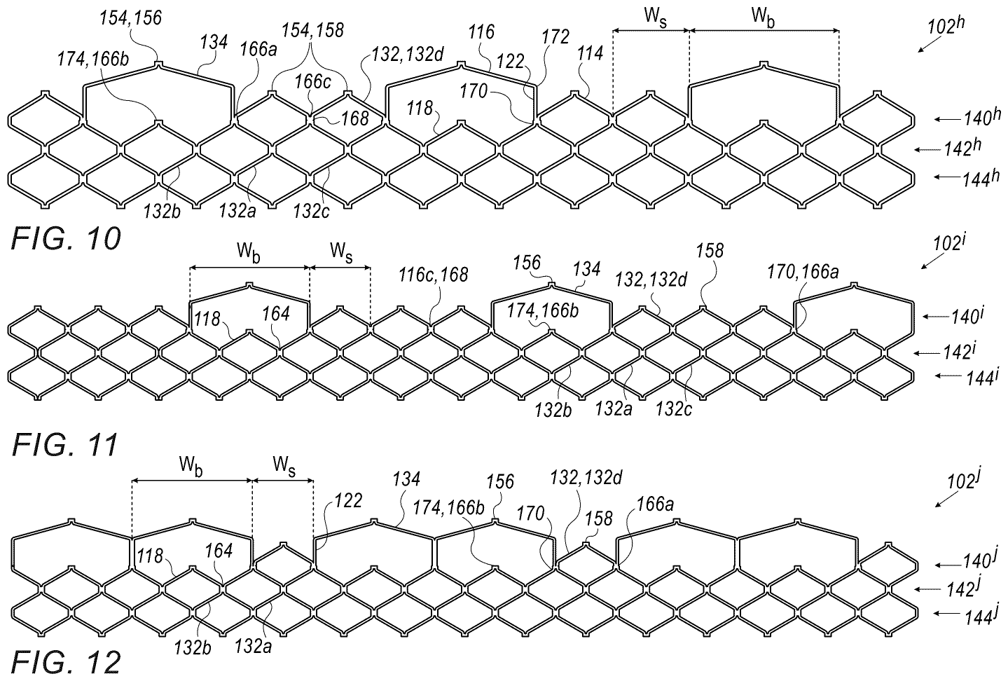

FIG. 10 shows an example of a frame 102^{h} of a prosthetic valve 100^{h}, which is similar in structure and function to frame 102^{c} described above with respect to FIG. 5, including second and third cell rows 142^{h}, 144^{h} comprising twelve narrow cells 132, but unlike frame 102^{c}, the first cell row 140^{h} of frame 102^{h} comprises three wide cells 134 and six narrow cells 132d, wherein the width Wb of the wide cells 134 of frame 102^{h} is twice as great as the width Ws of the narrow cells 132.

Each wide cell 134 of the exemplary frame 102^{h} illustrated in FIG. 10 includes two second-length proximal struts 116 diverging from a first outflow apex 156 towards two corresponding outflow vertical struts 122, and four distal struts 118 arranged in a zig-zagged pattern, which are also the first-length proximal struts 114 of narrow cells 132 of the second cell row 142^{h}.

Each of the six narrow cells 132d of first cell row 140^{h} includes two first-length proximal struts 114 diverging from the second outflow apex 158 towards two corresponding lateral junctions 168, wherein each narrow cell 132d is directly coupled to a wide cell 134 on one side thereof, and to another narrow cell 132d of the first cell row 140^{h} on its opposite side. Similarly, each wide cell 134 is directly coupled to a narrow cell 132d at each side thereof. This configuration results in two narrow cells 132d coupled to each other, disposed between each two consecutive wide cell 134 and coupled thereto. Each narrow cell 132d of the first cell row 140^{h} can be coupled to an outflow vertical strut 122, and in some examples, to a distal lateral junction 170 of the wide cell 134, as illustrated.

The second cell row 142^{h} includes six narrow cells 132a, three narrow cells 132b, and three narrow cells 132c. Each proximal junction 166a is disposed between a wide cell 134 and narrow cell 132d of the first cell row 140^{h}. Each proximal junction 166c of a narrow cell 132c is disposed between narrow cells 132d of the first cell row 140^{h}. Each free-ended proximal junction 166b is disposed within a corresponding wide cell 134, such that all three narrow cells 132b together define a total of three free junctions 174. In this manner, each free junction 174 is vertically aligned with the first outflow apex 156 of the same wide cell 134.

Each narrow cell 132b of the second cell row 142^{h} is directly connected to two narrow cell 132a via shared lateral junctions 168 on both sides thereof. Similarly, each narrow cell 132c of the second cell row 142^{h} is also directly connected to two narrow cell 132a via shared lateral junctions 168 on both sides thereof. Thus, each narrow cell 132a of the second cell row 142^{h} is directly coupled to a narrow cell 132b via a shared lateral junction 168 on one side thereof, and to a narrow cell 132c of the second cell row 142^{h} via a shared lateral junction 168 on its opposite side.

FIG. 11 shows an example of a frame 102ⁱ of a prosthetic valve 100ⁱ, which is similar in structure and function to frame 102^{d} described above with respect to FIGS. 6A-D, including second and third cell rows 142ⁱ, 144ⁱ comprising fifteen narrow cells 132, but unlike frame 102^{d}, the first cell row 140ⁱ of frame 102ⁱ comprises three wide cells 134 and nine narrow cells 132d, wherein the width Wb of the wide cells 134 of frame 102^{h} is twice as great as the width Ws of the narrow cells 132.

Each wide cell 134 of the exemplary frame 102ⁱ illustrated in FIG. 11 includes two second-length proximal struts 116 diverging from a first outflow apex 156 towards two corresponding outflow vertical struts 122, and four distal struts 118 arranged in a zig-zagged pattern, which are also the first-length proximal struts 114 of narrow cells 132 of the second cell row 142ⁱ.

Each of the nine narrow cells 132d of first cell row 140ⁱ includes two first-length proximal struts 114 diverging from the second outflow apex 158 towards two corresponding lateral junctions 168, wherein each narrow cell 132d is directly coupled to at least one other narrow cell 132d of the first cell row 140ⁱ via a mutual lateral junction 168. Each two consecutive wide cells 134 are separated from each other by a series of three narrow cells 132d. Thus, three narrow cells 132d of the first cell row 140ⁱ are each directly coupled to other two narrow cells 132d of the first cell row 140ⁱ via mutual lateral junction 168 on both sides thereof, while the remaining six cells 132d are each directly coupled to a wide cell 134 on one side, and to another narrow cell 132d of the first cell row 140ⁱ on the opposite side. Similarly, each wide cell 134 is directly coupled to a narrow cell 132d at each side thereof. A narrow cell 132d that is coupled to a wide cell 134 of the first cell row 140ⁱ, can be coupled to an outflow vertical strut 122, and in some examples, to a distal lateral junction 170 of the wide cell 134, as illustrated.

The second cell row 142ⁱ includes six narrow cells 132a, three narrow cells 132b, and six narrow cells 132c. Each proximal junction 166a is disposed between a wide cell 134 and narrow cell 132d of the first cell row 140ⁱ. Each proximal junction 166c of a narrow cell 132c is disposed between narrow cells 132d of the first cell row 140ⁱ. Each free-ended proximal junction 166b is disposed within a corresponding wide cell 134, such that all three narrow cells 132b together define a total of three free junctions 174. In this manner, each free junction 174 is vertically aligned with the first outflow apex 156 of the same wide cell 134.

Each narrow cell 132b of the second cell row 142ⁱ is directly connected to two narrow cell 132a via shared lateral junctions 168 on both sides thereof. Each narrow cell 132c of the second cell row 142ⁱ is directly coupled to a narrow cell 132b via a shared lateral junction 168 on one side thereof, and to another narrow cell 132c of the second cell row 142ⁱ via a shared lateral junction 168 on its opposite side. Thus, each narrow cell 132a of the second cell row 142ⁱ is directly coupled to a narrow cell 132b via a shared lateral junction 168 on one side thereof, and to a narrow cell 132c of the second cell row 142ⁱ via a shared lateral junction 168 on its opposite side.

FIG. 12 shows an example of a frame 102^{j} of a prosthetic valve 100^{j}, which is similar in structure and function to frame 102ⁱ described above with respect to FIG. 11, including second and third cell rows 142^{j}, 144^{j} comprising fifteen narrow cells 132, but unlike frame 102ⁱ, the first cell row 140^{j} of frame 102^{j} comprises six wide cells 134 and three narrow cells 132d, wherein the width Wb of the wide cells 134 of frame 102ⁱ is twice as great as the width Ws of the narrow cells 132.

Each wide cell 134 of the exemplary frame 102^{j} illustrated in FIG. 12 includes two second-length proximal struts 116 diverging from a first outflow apex 156 towards two corresponding outflow vertical struts 122, and four distal struts 118 arranged in a zig-zagged pattern, which are also the first-length proximal struts 114 of narrow cells 132 of the second cell row 142^{j}.

Each of the three narrow cells 132d of first cell row 140^{j} includes two first-length proximal struts 114 diverging from the second outflow apex 158 towards two corresponding lateral junctions 168, wherein each narrow cell 132d is directly coupled to two wide cell 134 on both sides thereof. Each two consecutive narrow cells 132d of first cell row 140^{j} are separated from each other by a couple of wide cell 134. Thus, each wide cell 134 is directly coupled to another wide cell 134 on one side thereof, for example via a shared outflow vertical strut 122, and to a narrow cell 132d of first cell row 140^{j} at the opposite side. Each narrow cell 132d can be coupled, on each side thereof, to an outflow vertical strut 122, and in some examples, to a distal lateral junction 170 of a corresponding wide cell 134, as illustrated.

The second cell row 142^{j} includes nine narrow cells 132a and six narrow cells 132b. Six of the narrow cells 132a have a proximal junction 166a disposed between a wide cell 134 and narrow cell 132d of the first cell row 140^{j}, while the other three narrow cells 132a have a proximal junction 166a disposed between two wide cells 134. Each free-ended proximal junction 166b is disposed within a corresponding wide cell 134, such that all six narrow cells 132b together define a total of six free junctions 174. In this manner, each free junction 174 is vertically aligned with the first outflow apex 156 of the same wide cell 134.

Each narrow cell 132b of the second cell row 142^{j} is directly connected to two narrow cell 132a via shared lateral junctions 168 on both sides thereof. Six of the narrow cells 132a of the second cell row 142^{j} are directly coupled, each, to a narrow cell 132b via a shared lateral junction 168 on one side, and to another narrow cell 132a of the second cell row 142^{j} via a shared lateral junction 168 on the other side. Each of the three remaining narrow cells 132a is directly connected to two narrow cells 132b via shared lateral junctions 168 on both sides thereof.

While the wide cells 134 in any of the examples illustrated in FIGS. 10-12 are shown to include outflow vertical struts 122, it is to be understood that in other examples, these wide cells do not necessarily include such vertical struts, such that they may share lateral junctions by which they are coupled either to the narrow cells 132d of the first cell rows, or to other wide cells 134 (for example, having two wide cells as those shown in FIG. 12, interconnected by a mutual lateral junction instead of a mutual outflow vertical strut).

In some examples, a frame that includes a plurality of narrow struts in the second and third cell rows, and a plurality of wide cells, with or without a plurality of narrow cells, in the first cells row, further comprises an intermediate cell row 138 disposed between the first cell row (140) and the second cell row (142), wherein unlike the first and second cell rows, in which every cell in each cell row is directly coupled to two other cells of the same cell row on both sides of the cell, the intermediate cell row 138 includes narrow cells 132—at least some of which are spaced from each other. In other words, at least some of the narrow cells 132 of the intermediate cell row 138 have at least one side which is not directly coupled to any adjacent narrow cell 132 of the same intermediate cell row 138.

Each wide cell 134 in such examples can include two distal struts 118 diverging from a distal junction 164 (for example, upward and sideways) towards intermediate junctions 175, and two interconnecting struts 119 extending from the intermediate junctions 175, generally in the same direction as that of the distal struts 118, towards lateral junctions (168) and/or outflow vertical struts (122) of the wide cell 134. In some examples, the distal struts 118 and interconnecting struts 119 may be formed as unitary continuous struts extending between a distal junction 164 and lateral junctions (168) and/or outflow vertical struts (122) of the wide cell 134, defining the intermediate junctions 175 there-between.

Each intermediate junction 175 disposed between the distal strut 118 and interconnecting strut 119 is also a lateral junction 168 of a narrow cell 132 of the intermediate cell row 138, and the interconnecting strut 119 is also a first-length proximal strut 114 of the same narrow cell 132 of the intermediate cell row 138. Thus, each wide cell 134 includes: (i) a distal junction 164 which is also an proximal junction 166 of a narrow cell 132 of the third cell row (144), as well as a lateral junction 168 between two narrow cells 132 of the second cell row (142); (ii) two distal struts 118 which are also first-length proximal struts 114 of narrow cells 132 of the second cell row (142); (iii) two intermediate junctions 175 which are also lateral junctions 168 of narrow cells 132 of the intermediate cell row 138, as well as proximal junctions 166 of narrow cells 132 of the second cell row (142); and (iv) two interconnecting struts 119 which are also first-length proximal struts 114 of narrow cells 132 of the intermediate cell row 138.

Depending on the shape and size of the intermediate junctions 175, the interconnecting strut 119 and distal strut 118 can extend along a common axis, or can be slightly offset relative to each other (i.e., provided with substantially parallel axes).

In some examples, narrow cells 132 can include several types of cells, including narrow cells 132a, 132b, 132c and 132d described above, as well as narrow cells 132e and 132f, all of which are identical in shape and size, but may differ by the surrounding cells they are attached to. A narrow cell 132e of any second cell row of the various examples of the current specification, defines an proximal junction 166e which is an intermediate junctions 175 of a corresponding wide cell 134. A narrow cell 132f of any third cell row of the various examples of the current specification, defines an proximal junction 166f which is the distal junction 164 of a corresponding wide cell 134.

FIG. 13 shows an example of a frame 102^{k} of a prosthetic valve 100^{k}, which is similar in structure and function to frame 102^{a} described above with respect to FIG. 3, including a first cell row 140^{k} comprising three wide cell 134, and second and third cell rows 142^{k}, 144^{k} each comprising six narrow cells 132, wherein the width Wb of the wide cells 134 of frame 102^{k} is twice as great as the width Ws of the narrow cells 132. However, unlike frame 102^{a}, the frame 102^{k} further comprises an intermediate cell row 138^{k} disposed between the first cell row 140^{k} and the second cell row 142^{k}, the intermediate cell row 138^{k} comprising three narrow cells 132a.

Each wide cell 134 of the exemplary frame 102^{k} illustrated in FIG. 13 includes two second-length proximal struts 116 diverging from an outflow apex 154 towards two corresponding outflow vertical struts 122, from which two interconnecting struts 119 further extend towards two distal struts 118, converging in turn to define a distal junction 164 vertically aligned with the outflow apex 154.

All three narrow cells 132a of the intermediate cell row 138^{k} are laterally spaced from each other, such that none of the cells 132a is directly coupled to any other adjacent narrow cell 132 of the same intermediate cell row 138^{k}.

In some examples, at least two narrow cells 132a of the intermediate cell row 138 are laterally spaced from each other at a distance which is at least as great as the width Ws of a narrow cell, and in the illustrated example of intermediate cell row 138^{k}, can be equal to the width Ws.

The second cell row 142^{k} includes six narrow cells 132e, each defining an proximal junction 166e which is also an intermediate junction 175 of a corresponding wide cell 134. As shown, the second cell row 142^{k} is devoid of narrow cells of the type 132b, such that the wide cells 134 of first cell row 140^{k} are devoid of free junctions (174). Thus, the bottom or inflow portion of such wide cells 134 does not include a zig-zagged pattern of distal struts 118, which affects the overall shape of the wide cell 134, and results in an overall height H1 of a cell 134 of frame 102^{k} which is greater than the equivalent height of such cells in wide cells illustrated in FIG. 3 for example.

In the example illustrated in FIG. 13, the frame 102^{k} is shown to further comprise a fourth cell row 146^{k}, which is substantially similar to the third cell row 144^{k}, including the exact same number of narrow cells 132 (i.e., six narrow cells in the illustrated example). It is to be understood that a fourth cell row 146^{k} is illustrated in FIG. 13 to demonstrate that such a fourth cell row can be added not merely to frame 102^{k}, but rather to any other example of a frame described herein with reference to any of the FIGS. 3-14. Moreover, it is to be understood that the fourth cell row 146^{k} need not be the lowest cell row defining inflow apices (160), and that additional cell rows, such as a fifth, sixth, seventh cell rows and so on, may be similarly added to any of the frames disclosed herein in conjunction with any of the FIGS. 3-14. Finally, the fourth cell row 146^{k} illustrated in FIG. 13 to include narrow cells 132 that unlike the frame illustrated in FIG. 2, do not necessarily include inflow vertical struts. However, in other examples, a third cell row, a fourth cell row, or any other lower cell row (e.g., fifth, sixth, seventh, and the like) of any of the frames disclosed herein in conjunction with any of the FIGS. 3-14, can be equipped with cells that do include inflow vertical struts.

It is to be understood that while one example of a frame that includes a first cell row (140) with a plurality of wide cells such that each wide cell (134) is directly connected to two adjacent wide cells in the same first cells row, and further includes an intermediate cell row (138), is demonstrated in FIG. 3 based on a configuration similar to that of frame 102^{a} of FIG. 3 (i.e., having three wide cells in the first cell row, and six narrow cells in each of the second and third cell rows) only by way of illustration, and that any of the frames 102^{b}, 102^{c}, 102^{d}, 102^{f} and 102^{g} described in conjunction with FIGS. 4-9 can be similarly modified in the same manner to include an intermediate cell row (138), mutatis mutandis.

FIG. 14 shows an example of a frame 102^{l} of a prosthetic valve 100^{l}, which is similar in structure and function to frame 102^{h} described above with respect to FIG. 10, including a first cell row 140^{l} comprising three wide cell 134 and six narrow cells 132d, and second and third cell rows 142^{l}, 144^{l} each comprising twelve narrow cells 132, wherein the width Wb of the wide cells 134 of frame 102^{l} is twice as great as the width Ws of the narrow cells 132. However, unlike frame 102^{h}, the frame 102^{l} further comprises an intermediate cell row 138^{l} disposed between the first cell row 140^{l} and the second cell row 142^{l}, the intermediate cell row 138^{l} comprising nine narrow cells 132.

Each wide cell 134 of the exemplary frame 102^{l} illustrated in FIG. 14 includes two second-length proximal struts 116 diverging from a first outflow apex 156 towards two corresponding outflow vertical struts 122, from which two interconnecting struts 119 further extend towards two distal struts 118, converging in turn to define a distal junction 164 vertically aligned with the outflow apex 154.

The intermediate cell row 138^{l} includes six narrow cells 132a, and three narrow cells 132c disposed between narrow cells 132a. Each narrow cell 132a of the intermediate cell row 138^{l} is directly coupled to a narrow cells 132c of the same intermediate cell row 138^{l} on one side thereof, for example via a mutual lateral junction 168, while it is not directly coupled to any adjacent cell of the same cell row on its opposite side, but rather defines a lateral junction 168 which is an intermediate junction 175 of a wide cell 134. Thus, each two narrow cells 132a defining opposite interconnecting struts 119 and intermediate junction 175 of the same wide cell 134, are spaced from each other, for example at a distance equal to the width Ws. Each narrow cell 132c of the intermediate cell row 138^{l} is directly coupled to two adjacent narrow cells 132a on both sides thereof.

The second cell row 142^{l} includes six narrow cells 132e and six narrow cells 132, wherein each narrow cell 132e defines a proximal junction 166e which is also an intermediate junction 175 of a corresponding wide cell 134. Each narrow cell 132e of the second cell row 142^{l} is directly coupled to another narrow cell 132e on one side, and to an adjacent narrow cell 132c of the same second cell row 142^{l} on the other side, for example via mutual lateral junctions 168. Each narrow cell 132c of the second cell row 142^{l}, in turn, is directly coupled to another narrow cell 132c on one side, and to an adjacent narrow cell 132e of the same second cell row 142^{l} on the other side.

As shown, the second cell row 142^{l} is devoid of narrow cells of the type 132b, such that the wide cells 134 of first cell row 140^{l} are devoid of free junctions (174). Thus, the bottom or inflow portion of such wide cells 134 does not include a zig-zagged pattern of distal struts 118, which affects the overall shape of the wide cell 134 in a similar manner described above in conjunction with FIG. 13, resulting in an overall height H1 of a cell 134 of frame 102^{l} which is greater than the equivalent height of such cells in wide cells illustrated in FIG. 10 for example.

It is to be understood that while one example of a frame that includes a first cell row (140) with a plurality of wide cells (134) and narrow cells (132) in the first cell row (140), further including an intermediate cell row (138), is demonstrated in FIG. 14 based on a configuration similar to that of frame 102^{h} of FIG. 10 (i.e., having three wide cells and six narrow cells in the first cell row, as well as twelve narrow cells in each of the second and third cell rows) only by way of illustration, and that any of the frames 102ⁱ and/or 102^{j} described in conjunction with FIGS. 11 and/or 12, respectively, can be similarly modified in the same manner to include an intermediate cell row (138), mutatis mutandis.

As mentioned above, adding an intermediate cell row 138 can advantageously result in an enlarged wide cell 134, having a height H1 defined between its outflow apex 154 and distal junction/s 164 which is greater than the height shown in alternative examples, such as those described above and illustrated throughout FIGS. 3-12. In fact, any wide cell 134 of a frame that includes an intermediate cell row 138, such as wide cells 134 shown to be devoid of free junction (174) in the examples illustrated in FIGS. 13 and 14, will have a height H1 which is greater by half the height H2 of narrow cells 132 (for example, of intermediate cell row 138) compared with the height H1 of cells 134 described above and illustrated throughout FIGS. 3-12.

While the examples illustrated throughout FIGS. 3-14 show struts of the wide cells 132, that can be second-length proximal struts 116 or outflow vertical struts 122, intersecting with proximal junctions 166 of narrow cells 132 of the second cell row 142, in other examples such struts 116, 122 of the wide cells 132 can intersect with lateral junctions 168 of narrow cells 132 of the second cell row 142. For example, the exemplary frames illustrated in FIGS. 3-14 show wide cells 134 having their outflow vertical struts 122 extending between proximal lateral junctions 172, from which the second-length proximal struts 116 extend, and proximal junctions 166 of narrow cells 132 of the second cell row 142, such as proximal junctions 166a of narrow cells 132a, such that the distal lateral junctions 170 of the outflow vertical struts 122 are also the proximal junctions 166 of narrow cells 132 of the second cell row 142. Wide cells that do not include outflow vertical struts can alternatively have lateral junctions, defined between second-length proximal struts and corresponding distal struts thereof, which are also the proximal junctions 166 of narrow cells 132 of the second cell row 142 (alternative examples not illustrated).

In some examples, the width Wb of the wide cells 134 is at least twice as great as the width Ws of the narrow cells 132, wherein at least one second-length proximal strut 116 or at least one outflow vertical strut 122 of each wide cell 134 intersects with a lateral junction 168 defined between two narrow cells 134 of the second cell row 142. In some examples, both second-length proximal struts 116 or both outflow vertical strut 122 of each wide cell 134 intersect with corresponding lateral junctions 168 of narrow cells 134 of the second cell row 142. In some examples, all of the proximal junctions 166 of the narrow cells 134 of the second cell row 142 are free-ended proximal junctions.

In some examples, the second cell row 142 comprises narrow cells of the type 132g, wherein a narrow cell type 132g is defined to include a free-ended proximal junction 166g, and wherein at least one lateral junction 168 at one side of the narrow cell 132g converges with a lateral junction of a wide cell 134, such as a distal lateral junction 170 of a corresponding outflow vertical strut 122. A free-ended proximal junction 166g, similar to free-ended proximal junction 166a, is defined as a proximal junction of a narrow cell 132 which is not attached to any strut of any other cell, and in particular, not attached to any strut of a wide cell 134. It is to be understood that the definition of not being attached to any strut of a different cells refers to any other struts which are not the same as the two first-length proximal struts 114 that converge to define the free-ended proximal junction 166g. In other words, distal struts 118 of the wide cell 134 which are also the proximal struts 114 of the narrow cell 132 are not considered to be other struts of cell 134. In comparison, second-length proximal struts 116 or outflow vertical struts 122 of wide cell 134 are regarded as other struts of cell 134, and will not extend from a free-ended proximal junction 166g.

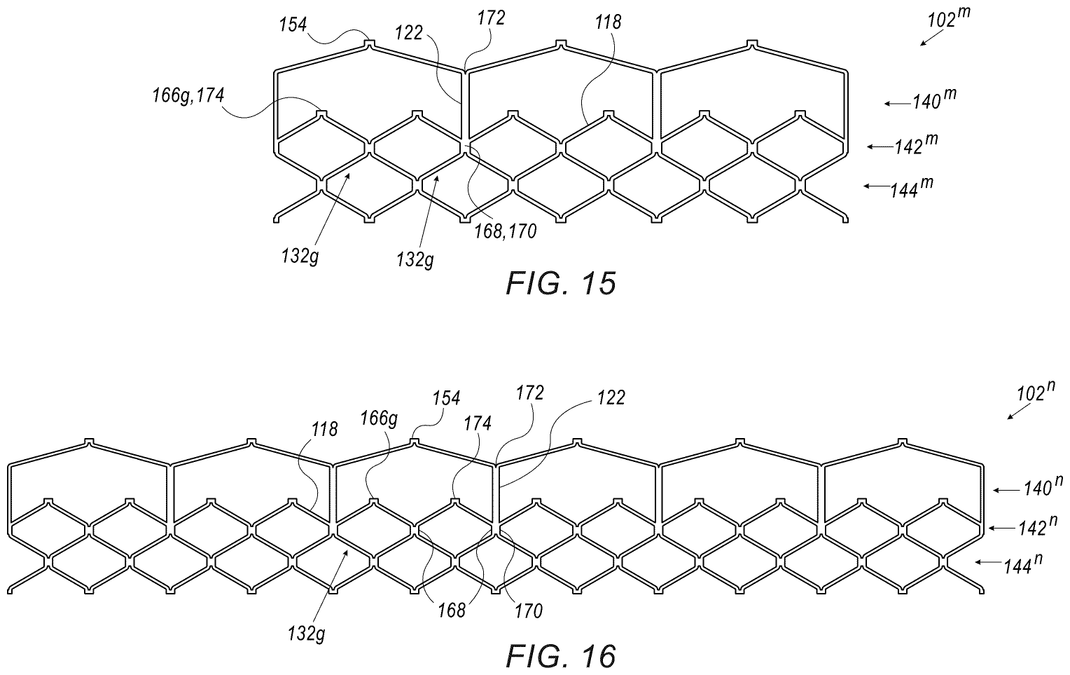

FIG. 15 shows an example of a frame 102^{m} of a prosthetic valve 100^{m}, which is similar in structure and function to frame 102^{a} of prosthetic valve 100^{a} disclosed above with respect to FIG. 3, including a first cell row 140^{m} having three wide cells 134 and each of the second and third cell rows 142^{m}, 144^{m} comprising six narrow cells 132, resulting in the width Wb of the wide cells 134 of frame 102^{m} being twice as great as the width Ws of the narrow cells 132. However, unlike frame 102^{a}, outflow vertical struts 122 of the wide cells 134 of the first cell row 140^{m} intersect with lateral junctions 168 of narrow cells 134 of the second cell row 142^{m}. In the illustrated example, all six cells of the second cell row 142^{m} are of the type 132g.

Each wide cell 134 of the exemplary frame 102^{m} illustrated in FIG. 15 includes two second-length proximal struts 116 diverging from an outflow apex 154 towards two corresponding outflow vertical struts 122, and four distal struts 118 arranged in a zig-zagged pattern, which are also the first-length proximal struts 114 of narrow cells 132 of the second cell row 142^{m}. In this manner, each such wide cell 134 has one distal junction 164, which is also a lateral junction 168 between narrow cells 132g of the second cell row 142^{m}, and two free junctions 174 which are also free-ended proximal junctions 166g of narrow cells 132g of the third cell row 144^{m}.

Each narrow cell 132g of the second cell row 142^{m} is directly connected to another narrow cell 132g via a shared lateral junction 168 on one side, and to another narrow cell 132g and a wide cell 134 of the first cell row 140^{m} via a shared lateral junction 168 on the opposite side which is also the distal lateral junction 170 of the wide cell 134. The outflow apex 154 is vertically aligned with its distal junction 164.

FIG. 16 shows an example of a frame 102ⁿ of a prosthetic valve 100ⁿ, which is similar in structure and function to frame 102^{m} of prosthetic valve 100^{m} disclosed above with respect to FIG. 15, except that each cell row 136ⁿ includes twice as much cells, and more specifically, the first cell row 140ⁿ includes six wide cells 134 and each of the second and third cell rows 142ⁿ, 144ⁿ includes twelve narrow cells 132.

It is to be understood that the two frames 102^{m} and 102ⁿ are illustrated by way of example only, and that any of the frames described above in conjunction with FIGS. 4-9 can be similarly modified to have other numbers of wide cells 134 and narrow cells 132, wherein struts of the wide cells 134 intersect with lateral junctions of narrow cells 132 of the second cell rows, as demonstrated for modifying frame 102^{a} of FIG. 3 to result in frame 102^{m} of FIG. 15, mutatis mutandis. Examples in which the number of narrow cells 132 in the second cell row 142 is higher than twice the number of wide cells 134 can include both narrow cells of type 132g and narrow cells of type 132a in the second cell row 142, such that all of the proximal junctions 166 of all narrow cells 132g, 132a of the second cell row 142 are free-ended proximal junctions 166g, 166a.

In some examples, a strut of a wide cell 134 can intersect with a lateral junction 168 of the second cell row 142 on one side, and another strut of the wide cell 134 can intersect with a proximal junction 166 of a narrow cell 132 of the second cell row 142 on the opposite side (examples not shown). In such cases, the width Wb of the wide cell 134, which is still wider than the width Ws, is not necessarily an integer multiple of the width Ws of narrow cell 132, but can rather be a non-integer multiple, such as the width Wb being at least 1.5 times as great the width Ws of narrow cell 132.

An inner space or opening of any example of a wide cell 134 described above with respect to FIGS. 3-16 is enclosed by two second-length proximal struts, two or more distal struts 118, optionally two outflow vertical struts 122, and optionally two interconnecting struts 119, together defining an outer perimeter of the wide cell 134. In some examples, none of the wide cells 134 described above includes any other additional struts disposed within the cell (i.e., internally, between the above-mentioned struts enclosing the wide cell's space), so as to ensure maximal open space that will allow undisturbed access to the coronary arteries therethrough.

FIG. 17 illustrates a delivery apparatus 200, according to an exemplary configuration, adapted to deliver a balloon expandable prosthetic valve 260 described herein (e.g., any of the exemplary prosthetic valves 100 described above with respect to FIGS. 3-16). It should be understood that the delivery apparatus 200 can be used to implant prosthetic devices other than prosthetic valves, such as stents or grafts.

The delivery apparatus 200 includes a handle 204 and a balloon catheter 252 having an inflatable balloon 250 mounted on its distal end. The prosthetic valve 260 can be carried in a crimped state over the balloon catheter 252. Optionally, an outer delivery shaft 224 can concentrically extend over the balloon catheter 252, and a push shaft 220 can be disposed over the balloon catheter 252, optionally between the balloon catheter 252 and the outer delivery shaft 224.

The outer delivery shaft 224, the push shaft 220, and the balloon catheter 252, can be configured to be axially movable relative to each other. For example, a proximally oriented movement of the outer delivery shaft 224 relative to the balloon catheter 252, or a distally oriented movement of the balloon catheter 252 relative to the outer delivery shaft 224, can expose the prosthetic valve 260 from the outer delivery shaft 224. The delivery apparatus 200 can further include a nosecone 240 carried by a nosecone shaft (hidden from view in FIG. 17) extending through a lumen of the balloon catheter 252.

The proximal ends of the balloon catheter 252, the outer delivery shaft 224, the push shaft 220, and optionally the nosecone shaft, can be coupled to the handle 204. During delivery of the prosthetic valve 260, the handle 204 can be maneuvered by an operator (e.g., a clinician or a surgeon) to axially advance or retract components of the delivery apparatus 200, such as the nosecone shaft, the balloon catheter 252, the outer delivery shaft 224, and/or the push shaft 220, through the patient's vasculature, as well as to inflate the balloon 250 mounted on the balloon catheter 252, so as to expand the prosthetic valve 260, and to deflate the balloon 250 and retract the delivery apparatus 200 once the prosthetic valve 260 is mounted in the implantation site.

The handle 204 can include a steering mechanism configured to adjust the curvature of the distal end portion of the delivery apparatus 200. In the illustrated example, the handle 204 includes an adjustment member, such as the illustrated rotatable knob 206a, which in turn is operatively coupled to the proximal end portion of a pull wire. The pull wire can extend distally from the handle 204 through the outer delivery shaft 224 and has a distal end portion affixed to the outer delivery shaft 224 at or near the distal end of the outer delivery shaft 224. Rotating the knob 206a can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein. The handle 204 can further include an adjustment mechanism including an adjustment member, such as the illustrated rotatable knob 206b. The adjustment mechanism can be configured to adjust the axial position of the push shaft 220 relative to the balloon catheter.

The prosthetic valve 260 can be carried by the delivery apparatus 200 during delivery in a crimped state, and expanded by balloon inflation to secure it in a native heart valve annulus. In an exemplary implantation procedure, the prosthetic valve 260 is initially crimped over the balloon catheter 252, proximal to the inflatable balloon 250. Because prosthetic valve 260 is crimped at a location different from the location of balloon 250, prosthetic valve 260 can be crimped to a lower profile than would be possible if it was crimped on top of balloon 250. This lower profile permits the clinician to more easily navigate the delivery apparatus 200 (including crimped prosthetic valve 260) through a patient's vasculature to the treatment location. The lower profile of the crimped prosthetic valve is particularly helpful when navigating through portions of the patient's vasculature which are particularly narrow, such as the iliac artery.

The balloon 250 can be secured to balloon catheter 252 at its balloon proximal end, and to either the balloon catheter 252 or the nosecone 240 at its distal end. The distal end portion of the push shaft 220 is positioned proximal to the outflow end (e.g., outflow end 104) of the prosthetic valve 260.

When reaching the site of implantation, and prior to balloon inflation, the push shaft 220 is advanced distally, allowing its distal end portion to contact and push against the outflow end of prosthetic valve 260, pushing the valve 260 distally therewith. The distal end of push shaft 220 is dimensioned to engage with the outflow end of the prosthetic valve 260 in a crimped configuration of the valve. In some implementations, the distal end portion of the push shaft 220 can be flared radially outward, to terminate at a wider-diameter that can contact the prosthetic valve 260 in its crimped state. Push shaft 220 can then be advanced distally, pushing the prosthetic valve 260 therewith, until the crimped prosthetic valve 260 is disposed around the balloon 250, at which point the balloon 250 can be inflated to radially expand the prosthetic valve 260. Once the prosthetic valve 260 is expanded to its functional diameter within a native annulus, the balloon 250 can be deflated, and the delivery apparatus 200 can be retrieved from the patient's body.

In particular implementations, the delivery apparatus 200 with the prosthetic valve 260 assembled thereon, can be packaged in a sterile package that can be supplied to end users for storage and eventual use. In particular implementations, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the prosthetic valve 260 and the delivery apparatus 200, can be free of any liquid. Methods for treating tissue leaflets for dry storage are disclosed in U.S. Pat. Nos. 8,007,992 and 8,357,387, both of which documents are incorporated herein by reference.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve, comprising:
a frame having an inflow end and an outflow end, wherein the frame is movable between a radially compressed and a radially expanded state, the frame comprising:
   a first cell row comprising a plurality of wide cells, wherein each wide cell defines an outflow apex and comprises two second-length proximal struts diverging therefrom, and wherein each wide cell of the first cell row is directly coupled to two other adjacent wide cells of the first cell row;
   a second cell row comprising a plurality of narrow cells, wherein each narrow cell of the second cell row is directly coupled to two adjacent narrow cells of the second cell row;
   a third cell row comprising a plurality of narrow cells, wherein each narrow cell of the third cell row is directly coupled to two adjacent narrow cells of the third cell row;
wherein the number of narrow cells in the second cell row is identical to the number of narrow cells in the third cells row;
wherein all of the narrow cells of the second cell row and the third cell row have the same height and the same width; and
wherein the width of each wide cell of the first cell row is at least three times as great as the width of any narrow cell.

Example 2. The prosthetic valve of any example herein, particularly example 1, further comprising a leaflet assembly mounted within the frame, the leaflet assembly comprising a plurality of leaflets arranged together to form commissures that are coupled to the frame.

Example 3. The prosthetic valve of any example herein, particularly any one of examples 1 or 2, further comprising an inner skirt secured to an inner surface of the frame.

Example 4. The prosthetic valve of any example herein, particularly any one of examples 1 to 3, further comprising an outer skirt secured to an outer surface of the frame.

Example 5. The prosthetic valve of any example herein, particularly any one of examples 1 to 4, wherein each wide cell comprises two outflow vertical struts extending from the second-length proximal struts, and wherein each wide cell is directly coupled to adjacent wide cells via mutual outflow vertical struts.

Example 6. The prosthetic valve of any example herein, particularly example 5, wherein at least some of the outflow vertical struts are commissure support struts, each commissure support strut defining a commissure window.

Example 7. The prosthetic valve of any example herein, particularly any one of examples 5 or 6, wherein each outflow vertical strut extends from an proximal junction of a corresponding narrow cell of the second cell row.

Example 8. The prosthetic valve of any example herein, particularly any one of examples 1 to 4, wherein each second-length proximal strut extends from an proximal junction of a corresponding narrow cell of the second cell row.

Example 9. The prosthetic valve of any example herein, particularly any one of examples 1 to 8, wherein the width of each wide cell row is three times as great as the width of any narrow cell.

Example 10. The prosthetic valve of any example herein, particularly example 9, wherein the plurality of wide cells comprises three wide cells in the first cell row, and wherein the plurality of narrow cells of each of the second and third cell rows comprises nine narrow cells in each of the second and third cell rows.

Example 11. The prosthetic valve of any example herein, particularly example 9, wherein the plurality of wide cells comprises six wide cells in the first cell row, and wherein the plurality of narrow cells of each of the second and third cell rows comprises eighteen narrow cells in each of the second and third cell rows.

Example 12. The prosthetic valve of any example herein, particularly any one of examples 9 to 11, wherein each wide cell comprises three distal junctions, two free junctions, and six distal struts arranged in a zig-zagged pattern between lateral junctions, the distal junctions and the free junctions of the wide cell.

Example 13. The prosthetic valve of any example herein, particularly example 12, wherein the outflow apex of each wide cell is vertically aligned with a middle-most distal junction of its three distal junctions.

Example 14. The prosthetic valve of any example herein, particularly any one of examples 1 to 8, wherein the width of each wide cell row is four times as great as the width of any narrow cell.

Example 15. The prosthetic valve of any example herein, particularly example 14, wherein the plurality of wide cells comprises three wide cells in the first cell row, and wherein the plurality of narrow cells of each of the second and third cell rows comprises twelve narrow cells in each of the second and third cell rows.

Example 16. The prosthetic valve of any example herein, particularly example 15, wherein each wide cell comprises four distal junctions, three free junctions, and eight distal struts arranged in a zig-zagged pattern between two lateral junctions, the distal junctions and the free junctions of the wide cell.

Example 17. The prosthetic valve of any example herein, particularly example 16, wherein the outflow apex of each wide cell is vertically aligned with a middle-most free junction of its three free junctions.

Example 18. The prosthetic valve of any example herein, particularly any one of examples 1 to 8, wherein the width of each wide cell row is five times as great as the width of any narrow cell.

Example 19. The prosthetic valve of any example herein, particularly example 18, wherein the plurality of wide cells comprises three wide cells in the first cell row, and wherein the plurality of narrow cells of each of the second and third cell rows comprises fifteen narrow cells in each of the second and third cell rows.

Example 20. The prosthetic valve of any example herein, particularly example 19, wherein each wide cell comprises five distal junctions, four free junctions, and ten distal struts arranged in a zig-zagged pattern between two lateral junctions, the distal junctions and the free junctions of the wide cell.

Example 21. The prosthetic valve of any example herein, particularly example 20, wherein the outflow apex of each wide cell is vertically aligned with a middle-most distal junction of its five distal junctions.

Example 22. The prosthetic valve of any example herein, particularly any one of examples 1 to 8, wherein the width of each wide cell row is six times as great as the width of any narrow cell.

Example 23. The prosthetic valve of any example herein, particularly example 22, wherein the plurality of wide cells comprises three wide cells in the first cell row, and wherein the plurality of narrow cells of each of the second and third cell rows comprises eighteen narrow cells in each of the second and third cell rows.

Example 24. The prosthetic valve of any example herein, particularly example 23, wherein each wide cell comprises six distal junctions, five free junctions, and twelve distal struts arranged in a zig-zagged pattern between two lateral junctions, the distal junctions and the free junctions of the wide cell.

Example 25. The prosthetic valve of any example herein, particularly example 24, wherein the outflow apex of each wide cell is vertically aligned with a middle-most free junction of its five free junctions.

Example 26. A prosthetic valve, comprising:
a frame having an inflow end and an outflow end, wherein the frame is movable between a radially compressed and a radially expanded state, the frame comprising:
   a first cell row comprising a plurality of cells, the plurality of cells of the first cell row comprising a plurality of wide cells and a plurality of narrow cells, wherein each wide cell defines a first outflow apex and comprises two second-length proximal struts diverging therefrom, wherein each narrow cell of the first cell row defines a second outflow apex and comprises two first-length proximal struts diverging therefrom;
   a second cell row comprising a plurality of narrow cells, wherein each narrow cell of the second cell row is directly coupled to two adjacent narrow cells of the second cell row;
   a third cell row comprising a plurality of narrow cells, wherein each narrow cell of the third cell row is directly coupled to two adjacent narrow cells of the third cell row;
wherein each cell of the first cell row is directly coupled to two other adjacent cells of the first cell row;
wherein the number of narrow cells in the second cell row is identical to the number of narrow cells in the third cells row;
wherein all of the narrow cells of the first cell row, the second cell row and the third cell row, have the same height and the same width; and
wherein the width of each wide cell of the first cell row is at least twice as great as the width of any narrow cell.

Example 27. The prosthetic valve of any example herein, particularly example 26, wherein the height of each wide cell is greater than the height of each narrow cell.

Example 28. The prosthetic valve of any example herein, particularly any one of examples 26 or 27, further comprising a leaflet assembly mounted within the frame, the leaflet assembly comprising a plurality of leaflets arranged together to form commissures that are coupled to the frame.

Example 29. The prosthetic valve of any example herein, particularly any one of examples 26 to 28, further comprising an inner skirt secured to an inner surface of the frame.

Example 30. The prosthetic valve of any example herein, particularly any one of examples 26 to 29, further comprising an outer skirt secured to an outer surface of the frame.

Example 31. The prosthetic valve of any example herein, particularly any one of examples 26 to 30, wherein each wide cell comprises two outflow vertical struts extending from the second-length proximal struts, and wherein each wide cell is directly coupled to adjacent wide cells via mutual outflow vertical struts.

Example 32. The prosthetic valve of any example herein, particularly example 31, wherein at least some of the outflow vertical struts are commissure support struts, each commissure support strut defining a commissure window.

Example 33. The prosthetic valve of any example herein, particularly any one of examples 26 to 32, wherein the width of each wide cell row is twice as great as the width of any narrow cell.

Example 34. The prosthetic valve of any example herein, particularly example 33, wherein the first cell row comprises three wide cells and six narrow cells, and wherein the plurality of narrow cells of each of the second and third cell rows comprises twelve narrow cells in each of the second and third cell rows.

Example 35. The prosthetic valve of any example herein, particularly example 33, wherein two narrow cells are disposed between each two consecutive wide cells in the first cell row.

Example 36. The prosthetic valve of any example herein, particularly example 33, wherein each narrow cell of the first cell row is directly coupled to one other narrow cell of the first cell row on one side, and to a wide cell on the opposite side.

Example 37. The prosthetic valve of any example herein, particularly example 33, wherein each wide cell is directly coupled to two adjacent narrow cells of the six narrow cells of the first cell row.

Example 38. The prosthetic valve of any example herein, particularly any one of examples 34 to 37, wherein each wide cell comprises two distal junctions, a single free junction, and four distal struts arranged in a zig-zagged pattern between two lateral junctions, the distal junctions and the free junction of the wide cell.

Example 39. The prosthetic valve of any example herein, particularly example 38, wherein the outflow apex of each wide cell is vertically aligned with its free junction.

Example 40. The prosthetic valve of any example herein, particularly any one of examples 34 to 37, wherein the frame further comprises an intermediate cell row disposed between the first cell row and the second cell row, wherein the intermediate cell row comprises nine narrow cells, and wherein at least one narrow cell of the intermediate cell row is connected to another narrow cell of the intermediate cell row on one side thereof, but is not connected to any other narrow cell of the intermediary cell row on its opposite side.

Example 41. The prosthetic valve of any example herein, particularly example 40, wherein each wide cell comprises a single distal junction vertically aligned with the first outflow apex of the wide cell.

Example 42. The prosthetic valve of any example herein, particularly example 41, wherein each wide cell comprises two distal struts diverging from the distal junction towards two intermediate junctions, and two interconnecting struts, each interconnecting strut further extending from a respective intermediate junction.

Example 43. The prosthetic valve of any example herein, particularly any one of examples 40 to 41, wherein the wide cells are devoid of free junctions.

Example 44. The prosthetic valve of any example herein, particularly example 33, wherein the first cell row comprises three wide cells and nine narrow cells, and wherein the plurality of narrow cells of each of the second and third cell rows comprises twelve narrow cells in each of the second and third cell rows.

Example 45. The prosthetic valve of any example herein, particularly example 33, wherein three narrow cells are disposed between each two consecutive wide cells in the first cell row.

Example 46. The prosthetic valve of any example herein, particularly example 33, wherein at least three narrow cell of the first cell row are directly coupled to two adjacent narrow cells of the first cell row.

Example 47. The prosthetic valve of any example herein, particularly example 33, wherein each wide cell is directly coupled to two adjacent narrow cells of the nine narrow cells of the first cell row.

Example 48. The prosthetic valve of any example herein, particularly any one of examples 44 to 47, wherein each wide cell comprises two distal junctions, a single free junction, and four distal struts arranged in a zig-zagged pattern between two lateral junctions, the distal junctions and the free junction of the wide cell.

Example 49. The prosthetic valve of any example herein, particularly example 48, wherein the outflow apex of each wide cell is vertically aligned with its free junction.

Example 50. The prosthetic valve of any example herein, particularly example 33, wherein the first cell row comprises six wide cells and three narrow cells, and wherein the plurality of narrow cells of each of the second and third cell rows comprises twelve narrow cells in each of the second and third cell rows.

Example 51. The prosthetic valve of any example herein, particularly example 33, wherein a single narrow cell is disposed between each two consecutive wide cells in the first cell row.

Example 52. The prosthetic valve of any example herein, particularly example 33, wherein each narrow cell of the first cell row is directly coupled to two adjacent wide cells.

Example 53. The prosthetic valve of any example herein, particularly example 33, wherein each wide cell is directly coupled to another adjacent wide cell on one side thereof, and to a narrow cell of the first cell row on its opposite side.

Example 54. The prosthetic valve of any example herein, particularly any one of examples 50 to 53, wherein each wide cell comprises two distal junctions, a single free junction, and four distal struts arranged in a zig-zagged pattern between two lateral junctions, the distal junctions and the free junction of the wide cell.

Example 55. The prosthetic valve of any example herein, particularly example 54, wherein the outflow apex of each wide cell is vertically aligned with its free junction.

Example 56. A prosthetic valve, comprising:
a frame having an inflow end and an outflow end, wherein the frame is movable between a radially compressed and a radially expanded state, the frame comprising:
   a first cell row comprising three wide cells, wherein each wide cell defines an outflow apex and comprises two second-length proximal struts diverging therefrom, and wherein each wide cell of the first cell row is directly coupled to two other adjacent wide cells of the first cell row;
   a second cell row comprising six narrow cells, wherein each narrow cell of the second cell row is directly coupled to two adjacent narrow cells of the second cell row;
   a third cell row comprising six narrow cells, wherein each narrow cell of the third cell row is directly coupled to two adjacent narrow cells of the third cell row;
wherein all of the narrow cells of the second cell row and the third cell row have the same height and the same width; and
wherein the width of each wide cell of the first cell row is at least twice as great as the width of any narrow cell.

Example 57. The prosthetic valve of any example herein, particularly example 56, further comprising a leaflet assembly mounted within the frame, the leaflet assembly comprising a plurality of leaflets arranged together to form commissures that are coupled to the frame.

Example 58. The prosthetic valve of any example herein, particularly any one of examples 56 or 57, further comprising an inner skirt secured to an inner surface of the frame.

Example 59. The prosthetic valve of any example herein, particularly any one of examples 56 to 58, further comprising an outer skirt secured to an outer surface of the frame.

Example 60. The prosthetic valve of any example herein, particularly any one of examples 56 to 59, wherein each wide cell comprises two outflow vertical struts extending from the second-length proximal struts, and wherein each wide cell is directly coupled to adjacent wide cells via mutual outflow vertical struts.

Example 61. The prosthetic valve of any example herein, particularly example 60, wherein at least some of the outflow vertical struts are commissure support struts, each commissure support strut defining a commissure window.

Example 62. The prosthetic valve of any example herein, particularly any one of examples 60 or 61, wherein each outflow vertical strut extends from an proximal junction of a corresponding narrow cell of the second cell row.

Example 63. The prosthetic valve of any example herein, particularly any one of examples 56 to 59, wherein each second-length proximal strut extends from an proximal junction of a corresponding narrow cell of the second cell row.

Example 64. The prosthetic valve of any example herein, particularly any one of examples 56 to 63, wherein each wide cell comprises two distal junctions, a single free junction, and four distal struts arranged in a zig-zagged pattern between two lateral junctions, the distal junctions and the free junction of the wide cell.

Example 65. The prosthetic valve of any example herein, particularly example 64, wherein the outflow apex of each wide cell is vertically aligned with its free junction.

Example 66. The prosthetic valve of any example herein, particularly any one of examples 56 to 64, wherein the frame further comprises an intermediate cell row disposed between the first cell row and the second cell row, wherein the intermediate cell row comprises three narrow cells, and wherein none of the narrow cells of the intermediate cell row is directly connected to any other narrow cell of the intermediate cell row.

Example 67. The prosthetic valve of any example herein, particularly example 66, wherein each wide cell comprises a single distal junction vertically aligned with the first outflow apex of the wide cell.

Example 68. The prosthetic valve of any example herein, particularly example 67, wherein each wide cell comprises two distal struts diverging from the distal junction towards two intermediate junctions, and two interconnecting struts, each interconnecting strut further extending from a respective intermediate junction.

Example 69. The prosthetic valve of any example herein, particularly any one of examples 66 to 68, wherein the wide cells are devoid of free junctions.

Example 70. A prosthetic valve, comprising:
a frame having an inflow end and an outflow end, wherein the frame is movable between a radially compressed and a radially expanded state, the frame comprising:
   a first cell row comprising nine wide cells, wherein each wide cell defines an outflow apex and comprises two second-length proximal struts diverging therefrom, and wherein each wide cell of the first cell row is directly coupled to two other adjacent wide cells of the first cell row,
   a second cell row comprising eighteen narrow cells, wherein each narrow cell of the second cell row is directly coupled to two adjacent narrow cells of the second cell row;
   a third cell row comprising eighteen narrow cells, wherein each narrow cell of the third cell row is directly coupled to two adjacent narrow cells of the third cell row;
wherein all of the narrow cells of the second cell row and the third cell row have the same height and the same width; and
wherein the width of each wide cell of the first cell row is at least twice as great as the width of any narrow cell.

Example 71. The prosthetic valve of any example herein, particularly example 70, further comprising a leaflet assembly mounted within the frame, the leaflet assembly comprising a plurality of leaflets arranged together to form commissures that are coupled to the frame.

Example 72. The prosthetic valve of any example herein, particularly any one of examples 70 or 71, further comprising an inner skirt secured to an inner surface of the frame.

Example 73. The prosthetic valve of any example herein, particularly any one of examples 70 to 72, further comprising an outer skirt secured to an outer surface of the frame

Example 74. The prosthetic valve of any example herein, particularly any one of examples 70 to 73, wherein each wide cell comprises two outflow vertical struts extending from the second-length proximal struts, and wherein each wide cell is directly coupled to adjacent wide cells via mutual outflow vertical struts.

Example 75. The prosthetic valve of any example herein, particularly example 74, wherein at least some of the outflow vertical struts are commissure support struts, each commissure support strut defining a commissure window.

Example 76. The prosthetic valve of any example herein, particularly any one of examples 74 or 75, wherein each outflow vertical strut extends from an proximal junction of a corresponding narrow cell of the second cell row.

Example 77. The prosthetic valve of any example herein, particularly any one of examples 70 to 73, wherein each second-length proximal strut extends from an proximal junction of a corresponding narrow cell of the second cell row.

Example 78. The prosthetic valve of any example herein, particularly any one of examples 70 to 77, wherein each wide cell comprises two distal junctions, a single free junction, and four distal struts arranged in a zig-zagged pattern between two lateral junctions, the distal junctions and the free junction of the wide cell.

Example 79. The prosthetic valve of any example herein, particularly example 78, wherein the outflow apex of each wide cell is vertically aligned with its free junction.

Example 80. The prosthetic valve of any example herein, particularly any one of examples 70 to 77, wherein the frame further comprises an intermediate cell row disposed between the first cell row and the second cell row, wherein the intermediate cell row comprises nine narrow cells, and wherein none of the narrow cells of the intermediate cell row is directly connected to any other narrow cell of the intermediate cell row.

Example 81. The prosthetic valve of any example herein, particularly example 80, wherein each wide cell comprises a single distal junction vertically aligned with the first outflow apex of the wide cell . . .

Example 82. The prosthetic valve of any example herein, particularly example 81, wherein each wide cell comprises two distal struts diverging from the distal junction towards two intermediate junctions, and two interconnecting struts, each interconnecting strut further extending from a respective intermediate junction.

Example 83. The prosthetic valve of any example herein, particularly any one of examples 80 to 82, wherein the wide cells are devoid of free junctions.

Example 84. A prosthetic valve, comprising:
a frame having an inflow end and an outflow end, wherein the frame is movable between a radially compressed and a radially expanded state, the frame comprising:
   a first cell row comprising a plurality of wide cells, wherein each wide cell defines an outflow apex and comprises two second-length proximal struts diverging therefrom, and wherein each wide cell of the first cell row is directly coupled to two other adjacent wide cells of the first cell row;
   a second cell row comprising a plurality of narrow cells, wherein each narrow cell of the second cell row is directly coupled to two adjacent narrow cells of the second cell row, and wherein each narrow cell of the second cell row comprises two first-length proximal struts converging at a free-ended proximal junction;
   a third cell row comprising a plurality of narrow cells, wherein each narrow cell of the third cell row is directly coupled to two adjacent narrow cells of the third cell row;
wherein the number of narrow cells in the second cell row is identical to the number of narrow cells in the third cells row;
wherein all of the narrow cells of the second cell row and the third cell row have the same height and the same width; and
wherein the width of each wide cell of the first cell row is at least twice as great as the width of any narrow cell.

Example 85. The prosthetic valve of any example herein, particularly example 84, further comprising a leaflet assembly mounted within the frame, the leaflet assembly comprising a plurality of leaflets arranged together to form commissures that are coupled to the frame.

Example 86. The prosthetic valve of any example herein, particularly any one of examples 84 or 85, further comprising an inner skirt secured to an inner surface of the frame.

Example 87. The prosthetic valve of any example herein, particularly any one of examples 84 to 86, further comprising an outer skirt secured to an outer surface of the frame.

Example 88. The prosthetic valve of any example herein, particularly any one of examples 84 to 87, wherein each wide cell comprises two outflow vertical struts extending from the second-length proximal struts, and wherein each wide cell is directly coupled to adjacent wide cells via mutual outflow vertical struts.

Example 89. The prosthetic valve of any example herein, particularly example 88, wherein at least some of the outflow vertical struts are commissure support struts, each commissure support strut defining a commissure window.

Example 90. The prosthetic valve of any example herein, particularly any one of examples 88 or 89, wherein each outflow vertical strut extends from a lateral junction of a corresponding narrow cell of the second cell row.

Example 91. The prosthetic valve of any example herein, particularly any one of examples 84 to 87, wherein each second-length proximal strut extends from a lateral junction of a corresponding narrow cell of the second cell row.

Example 92. The prosthetic valve of any example herein, particularly any one of examples 84 to 91, wherein the width of each wide cell row is twice as great as the width of any narrow cell.

Example 93. The prosthetic valve of any example herein, particularly example 92, wherein the plurality of wide cells comprises three wide cells in the first cell row, and wherein the plurality of narrow cells of each of the second and third cell rows comprises six narrow cells in each of the second and third cell rows.

Example 94. The prosthetic valve of any example herein, particularly example 92, wherein the plurality of wide cells comprises six wide cells in the first cell row, and wherein the plurality of narrow cells of each of the second and third cell rows comprises twelve narrow cells in each of the second and third cell rows.

Example 95. The prosthetic valve of any example herein, particularly example 92, wherein the plurality of wide cells comprises nine wide cells in the first cell row, and wherein the plurality of narrow cells of each of the second and third cell rows comprises eighteen narrow cells in each of the second and third cell rows.

Example 96. The prosthetic valve of any example herein, particularly any one of examples 92 to 95, wherein each wide cell comprises a single distal junction, two free junctions, and four distal struts arranged in a zig-zagged pattern between two lateral junctions, the distal junction and the free junctions of the wide cell.

Example 97. The prosthetic valve of any example herein, particularly example 96, wherein the outflow apex of each wide cell is vertically aligned with its distal junction.

Example 98. The prosthetic valve of any example herein, particularly any one of examples 84 to 91, wherein the width of each wide cell row is three times as great as the width of any narrow cell.

Example 99. The prosthetic valve of any example herein, particularly example 98, wherein the plurality of wide cells comprises three wide cells in the first cell row, and wherein the plurality of narrow cells of each of the second and third cell rows comprises nine narrow cells in each of the second and third cell rows.

Example 100. The prosthetic valve of any example herein, particularly example 98, wherein the plurality of wide cells comprises six wide cells in the first cell row, and wherein the plurality of narrow cells of each of the second and third cell rows comprises eighteen narrow cells in each of the second and third cell rows.

Example 101. The prosthetic valve of any example herein, particularly any one of examples 98 to 100, wherein each wide cell comprises two distal junctions, three free junctions, and six distal struts arranged in a zig-zagged pattern between lateral junctions, the distal junctions and the free junctions of the wide cell.

Example 102. The prosthetic valve of any example herein, particularly example 101, wherein the outflow apex of each wide cell is vertically aligned with a middle-most free junction of its three free junctions.

Example 103. The prosthetic valve of any example herein, particularly any one of examples 84 to 91, wherein the width of each wide cell row is four times as great as the width of any narrow cell.

Example 104. The prosthetic valve of any example herein, particularly example 103, wherein the plurality of wide cells comprises three wide cells in the first cell row, and wherein the plurality of narrow cells of each of the second and third cell rows comprises twelve narrow cells in each of the second and third cell rows.

Example 105. The prosthetic valve of any example herein, particularly example 104, wherein each wide cell comprises three distal junctions, four free junctions, and eight distal struts arranged in a zig-zagged pattern between two lateral junctions, the distal junctions and the free junctions of the wide cell.

Example 106. The prosthetic valve of any example herein, particularly example 105, wherein the outflow apex of each wide cell is vertically aligned with a middle-most distal junction of its three distal junctions.

Example 107. The prosthetic valve of any example herein, particularly any one of examples 84 to 91, wherein the width of each wide cell row is five times as great as the width of any narrow cell.

Example 108. The prosthetic valve of any example herein, particularly example 107, wherein the plurality of wide cells comprises three wide cells in the first cell row, and wherein the plurality of narrow cells of each of the second and third cell rows comprises fifteen narrow cells in each of the second and third cell rows.

Example 109. The prosthetic valve of any example herein, particularly example 108, wherein each wide cell comprises four distal junctions, five free junctions, and ten distal struts arranged in a zig-zagged pattern between two lateral junctions, the distal junctions and the free junctions of the wide cell.

Example 110. The prosthetic valve of any example herein, particularly example 109, wherein the outflow apex of each wide cell is vertically aligned with a middle-most free junction of its five free junctions.

Example 111. The prosthetic valve of any example herein, particularly any one of examples 84 to 91, wherein the width of each wide cell row is six times as great as the width of any narrow cell.

Example 112. The prosthetic valve of any example herein, particularly example 111, wherein the plurality of wide cells comprises three wide cells in the first cell row, and wherein the plurality of narrow cells of each of the second and third cell rows comprises eighteen narrow cells in each of the second and third cell rows.

Example 113. The prosthetic valve of any example herein, particularly example 112, wherein each wide cell comprises five distal junctions, six free junctions, and twelve distal struts arranged in a zig-zagged pattern between two lateral junctions, the distal junctions and the free junctions of the wide cell.

Example 114. The prosthetic valve of any example herein, particularly example 113, wherein the outflow apex of each wide cell is vertically aligned with a middle-most distal junction of its five distal junctions.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve, comprising:
a frame having an inflow end and an outflow end, wherein the frame is movable between a radially compressed and a radially expanded state, the frame comprising:
a first cell row comprising a plurality of wide cells, wherein each wide cell defines a single outflow apex and comprises two second-length proximal struts diverging therefrom, and wherein each wide cell of the first cell row is directly coupled to two other adjacent wide cells of the first cell row;
a second cell row comprising a plurality of narrow cells, wherein each narrow cell of the second cell row is directly coupled to two adjacent narrow cells of the second cell row;
a third cell row comprising a plurality of narrow cells, wherein each narrow cell of the third cell row is directly coupled to two adjacent narrow cells of the third cell row;
wherein the number of narrow cells in the second cell row is identical to the number of narrow cells in the third cells row;
wherein all of the narrow cells of the second cell row and the third cell row have the same height and the same width; and
wherein the width of each wide cell of the first cell row is at least three times as great as the width of any narrow cell.

2. The prosthetic valve of claim 1, wherein each wide cell comprises two outflow vertical struts extending from the second-length proximal struts, and wherein each wide cell is directly coupled to adjacent wide cells via mutual outflow vertical struts.

3. The prosthetic valve of claim 1 or 2, wherein the width of each wide cell row is three times as great as the width of any narrow cell.

4. The prosthetic valve of claim 3, wherein the plurality of wide cells comprises three wide cells in the first cell row, and wherein the plurality of narrow cells of each of the second and third cell rows comprises nine narrow cells in each of the second and third cell rows.

5. The prosthetic valve of claim 3, wherein the plurality of wide cells comprises six wide cells in the first cell row, and wherein the plurality of narrow cells of each of the second and third cell rows comprises eighteen narrow cells in each of the second and third cell rows.

6. The prosthetic valve of any one of claims 3 to 5, wherein each wide cell comprises three distal junctions, two free junctions, and six distal struts arranged in a zig-zagged pattern between the distal junctions and the free junctions of the wide cell.

7. The prosthetic valve of claim 6, wherein the outflow apex of each wide cell is vertically aligned with a middle-most distal junction of its three distal junctions.

8. The prosthetic valve of claim 1 or 2, wherein the width of each wide cell row is four times as great as the width of any narrow cell.

9. The prosthetic valve of claim 1 or 2, wherein the width of each wide cell row is five times as great as the width of any narrow cell.

10. The prosthetic valve of claim 1 or 2, wherein the width of each wide cell row is six times as great as the width of any narrow cell.

11. A prosthetic valve, comprising:
a frame having an inflow end and an outflow end, wherein the frame is movable between a radially compressed and a radially expanded state, the frame comprising:
a first cell row comprising a plurality of cells, the plurality of cells of the first cell row comprising a plurality of wide cells and a plurality of narrow cells, wherein each wide cell defines a single first outflow apex and comprises exactly two second-length proximal struts diverging therefrom, wherein each narrow cell of the first cell row defines a second outflow apex and comprises two first-length proximal struts diverging therefrom;
a second cell row comprising a plurality of narrow cells, wherein each narrow cell of the second cell row is directly coupled to two adjacent narrow cells of the second cell row;
a third cell row comprising a plurality of narrow cells, wherein each narrow cell of the third cell row is directly coupled to two adjacent narrow cells of the third cell row;
wherein each cell of the first cell row is directly coupled to two other adjacent cells of the first cell row;
wherein the number of narrow cells in the second cell row is identical to the number of narrow cells in the third cells row;
wherein all of the narrow cells of the first cell row, the second cell row and the third cell row, have the same height and the same width; and
wherein the width of each wide cell of the first cell row is at least twice as great as the width of any narrow cell.

12. The prosthetic valve of claim 11, wherein the height of each wide cell is greater than the height of each narrow cell.

13. The prosthetic valve of claim 11 or 12, wherein the width of each wide cell row is twice as great as the width of any narrow cell.

14. The prosthetic valve of claim 13, wherein the first cell row comprises three wide cells and six narrow cells, and wherein the plurality of narrow cells of each of the second and third cell rows comprises twelve narrow cells in each of the second and third cell rows.

15. The prosthetic valve of claim 14, wherein the frame further comprises an intermediate cell row disposed between the first cell row and the second cell row, wherein the intermediate cell row comprises nine narrow cells, and wherein at least one narrow cell of the intermediate cell row is connected to another narrow cell of the intermediate cell row on one side thereof, but is not connected to any other narrow cell of the intermediary cell row on its opposite side.

16. The prosthetic valve of claim 13, wherein the first cell row comprises three wide cells and nine narrow cells, and wherein the plurality of narrow cells of each of the second and third cell rows comprises twelve narrow cells in each of the second and third cell rows.

17. The prosthetic valve of claim 13, wherein the first cell row comprises six wide cells and three narrow cells, and wherein the plurality of narrow cells of each of the second and third cell rows comprises twelve narrow cells in each of the second and third cell rows.

18. A prosthetic valve, comprising:
a frame having an inflow end and an outflow end, wherein the frame is movable between a radially compressed and a radially expanded state, the frame comprising:
a first cell row comprising three wide cells, wherein each wide cell defines a single outflow apex and comprises exactly two second-length proximal struts diverging therefrom, and wherein each wide cell of the first cell row is directly coupled to two other adjacent wide cells of the first cell row;
a second cell row comprising six narrow cells, wherein each narrow cell of the second cell row is directly coupled to two adjacent narrow cells of the second cell row;
a third cell row comprising six narrow cells, wherein each narrow cell of the third cell row is directly coupled to two adjacent narrow cells of the third cell row; and
an intermediate cell row disposed between the first cell row and the second cell row, the intermediate cell row comprising three narrow cells;
wherein all of the narrow cells of the second cell row and the third cell row have the same height and the same width;
wherein the width of each wide cell of the first cell row is at least twice as great as the width of any narrow cell; and
wherein none of the narrow cells of the intermediate cell row is directly connected to any other narrow cell of the intermediate cell row.

19. The prosthetic valve of claim 18, wherein each wide cell comprises a single distal junction vertically aligned with the first outflow apex of the wide cell.

20. The prosthetic valve of claim 19, wherein each wide cell comprises two distal struts diverging from the distal junction towards two intermediate junctions, and two interconnecting struts, each interconnecting strut further extending from a respective intermediate junction.

21. The prosthetic valve of any one of claims 18 to 20, wherein the wide cells are devoid of free junctions.
